SEQUENCE LISTING (1 sequence)

SEQ ID NO: 1 - DNA, 26 bp, synthetic construct
  source 1..26: mol_type = other DNA
ccgggcaatgtcccgccgcaaacagg

(19) United States
(12) Patent Application Publication
(10) Pub. No.: US 2026/0287587 A1
(43) Pub. Date: Sep. 24, 2026

(54) BCL11B AS A BIOMARKER FOR BREAST CANCER PATIENTS TO IMPROVE CHEMOTHERAPY EFFICACY UTILIZING TNF-ALPHA TREATMENT

(71) Applicant: The Board of Trustees of the Leland Stanford Junior University, Stanford, CA (US)

(72) Inventors: Michael F. Clarke, Menlo Park, CA (US); Zhen Qi, Redwood City, CA (US)

(21) Appl. No.: 19/417,953

(22) Filed: Dec. 12, 2025

Related U.S. Application Data
(60) Provisional application No. 63/733,912, filed on Dec. 13, 2024.

(51) Int. Cl. G01N 33/57515; A61K 31/015; A61K 31/675; A61K 31/704; A61K 39/395; A61P 35/00; C12Q 1/6886

(52) U.S. Cl. CPC G01N 33/57515; A61K 31/015; A61K 31/675; A61K 31/704; A61K 39/3955; A61P 35/00; C12Q 1/6886; C12Q 2600/106; C12Q 2600/158

(57) ABSTRACT

Disclosed herein is the identification of the biomarker, BCL11B, in breast cancer, which is associated with an increased risk of relapse after chemotherapy. In an embodiment, a method of identifying a subset of breast cancer patients at high risk of developing chemotherapy resistance is provided, the method comprising determining the prevalence of BCL11B expression in a population of breast cancer cells, where higher levels of BCL11B expressing cells identify an individual with a higher risk of resistance when receiving chemotherapy. Individuals may be treated accordingly to decrease the risk of relapse.

### CROSS-REFERENCE TO RELATED APPLICATIONS

This application is a U.S. Application which application claims the benefit of U.S. Provisional Application No. 63/733,912, filed Dec. 13, 2024, which is incorporated herein by reference in their entirety.

### GOVERNMENT SUPPORT RESEARCH

This invention was made with Government support under contract CA255450 awarded by the National Institutes of Health and contracts W81XWH-13-1-0281 and W81SWH0110109287 awarded by the Department of Defense. The Government has certain rights in the invention.

### INCORPORATION BY REFERENCE OF SEQUENCE LISTING PROVIDED AS A SEQUENCE LISTING XML FILE

A Sequence Listing is provided herewith as a Sequence Listing XML, STAN-2233_seqlist, created on Jun. 8, 2026 and having a size of 2089 bytes. The contents of the Sequence Listing XML are incorporated herein by reference in their entirety.

### BACKGROUND

Systemic chemotherapy remains one of the principal modes of treatment for malignant tumors. Despite initially responding to chemotherapeutic drugs, many tumors develop resistance in a complex and unpredictable manner, making it one of the most challenging problems in cancer treatment. A prevailing adaptive mechanism explaining non-genetic resistance in several cancer types including colorectal cancer and breast cancer, is the theory of drug-tolerant persister, wherein a small population of cancer cells can enter a reversible drug-tolerant persister state to evade treatment. It has been conventionally suggested that tumor cells possess an equal capacity to enter the drug-tolerant state through transcriptional modulation upon treatment. However, the diverse responses of tumors to chemotherapy treatment observed in the clinic, which can range from complete to minimal, suggest that the emergence of drug resistance is likely predetermined by biological and molecular factors that remain largely elusive.

Growing evidence suggests that tumors are heterogeneous entities comprising different cell lineages or cell states with intrinsic variations in biological features. Thus, tumor subpopulations with pre-existing fitness advantages may preferentially adopt a drug-resistant state following treatment. Consistent with this concept, recent lineage-tracing studies in colorectal cancer provide direct evidence that certain pre-existing tumor subpopulations selectively persist during chemotherapy and drive regeneration once treatment is discontinued. Therefore, understanding how functionally distinct tumor subpopulations pre-existing in tumors respond to chemotherapy is essential for identifying the origin of chemoresistance and uncovering biomarkers that can predict drug responses, particularly in breast cancer, where such biomarkers are largely lacking.

Breast cancer is a highly heterogeneous disease that encompasses multiple subtypes with different clinical outcomes. Breast cancers are classified into three classical subtypes determined by the presence of the estrogen receptor (ER), progesterone receptor (PR), and the epidermal growth factor receptor HER2. Based on gene expression profiling, breast cancers can be stratified into several molecular subtypes: luminal-like (LumA and LumB), HER2-overexpressing, basal-like, normal-like, and claudin-low. Adding to the complexity, cancer cell populations in distinct cell states can co-exist in a single tumor, reflecting the normal mammary hierarchy. The normal mammary epithelium comprises an inner luminal cell layer and an outer basal cell layer and contains various cell states, including stem cells, luminal and basal progenitors, and mature luminal and myoepithelial cells. Breast tumors are thought to originate from different epithelial compartments and are often populated with multiple epithelial cell states. However, it is not clear how the initial cell state heterogeneity influences the drug responses in breast cancer. The epithelial-to-mesenchymal transition (EMT) has been shown to promote epithelial tumor cells, including breast cancer cells to adopt a mesenchymal cell state and gain resistance to therapies under certain conditions. However, EMT appears to be a rare event in vivo and the pathological evidence of EMT in human carcinoma samples has not been well established. Therefore, it is important to investigate whether there are pre-existing epithelial populations that are intrinsically resistant to chemotherapy and to determine strategies to prevent the development of drug resistance from these cells.

The present disclosure addresses the heterogeneity of breast cancers, and provides markers and therapeutic modalities for treatment.

### SUMMARY

Disclosed herein is the identification of the biomarker, BCL11B, in breast cancer, which is associated with an increased risk of relapse after chemotherapy. In an embodiment, a method of identifying a subset of breast cancer patients at high risk of developing chemotherapy resistance is provided, the method comprising determining the prevalence of BCL11B expression in a population of breast cancer cells, where a prevalence of greater than about 10%, greater than about 15%, greater than about 20% of BCL11B⁺ cells in the population identifies an individual with a higher risk of resistance when receiving chemotherapy.

In an embodiment, an individual with breast cancer identified as having a higher risk of resistance when receiving chemotherapy by having a higher prevalence of BCL11B⁺ cells is treated with a combination of an effective dose of a TNFα agonist, and a chemotherapeutic agent and/or an immunotherapy agent. Targeting pre-existing drug-tolerant cells with TNF-α treatment provides an approach to overcoming chemotherapy and/or immunotherapy resistance in breast cancer. In some embodiments the TNFα agonist is recombinant human TNFα. In some embodiments the chemotherapy comprises administration of an effective dose of an anthracycline, e.g. daunorubicin, doxorubicin, epirubicin, etc. In some embodiments the chemotherapy comprises administration of doxorubicin. In some embodiments doxorubicin is combined with cyclophosphamide. In some embodiments the chemotherapy comprises administration of a taxane.

In one embodiment of the invention, the methods of determining propensity to develop resistance to chemotherapy in breast cancer comprise obtaining a patient sample comprising breast cancer cells, e.g. a biopsy sample. The sample may be obtained prior to treatment, e.g. prior to radiation, prior to chemotherapy. The sample may be obtained following treatment, e.g. in a follow-up biopsy. The breast cancer cells, or biomolecules derived therefrom may be physically contacted with an affinity reagent specific for BCL11B. In some embodiments the affinity reagent is an antibody specific for BCL11B. Suitable methods of analysis include, without limitation, flow cytometry, mass cytometry, confocal microscopy, immunochemistry and the like. In other embodiments the sample is subjected to RNA analysis, e.g. RT-PCR, qPCR, RT-qPCR, RNA sequencing, etc. The number of cancer cells positive for expression of BCL11B is determined. The data is compared to measurements from a baseline cell population, e.g. a normal control, non-tumor sample from the individual, etc. The data can be normalized for comparison

In other embodiments of the invention a device or kit is provided for the analysis of patient samples. Such devices or kits will include reagents that specifically identify expression of BCL11B, including without limitation affinity reagents, PCR primers, etc. The reagents can be provided in isolated form, or pre-mixed with reagents suitable for the methods of the invention. A kit can include instructions for using the reagents to determine data from the sample; and instructions for statistically analyzing the data. Such a system may include a software component configured for analysis of data obtained by the methods of the invention.

In an embodiment, the method further comprises selecting a treatment regimen for the patient based on the analysis. Treatment regimens of interest can include decision-making for treating the patient with a combination of an effective dose of TNFα, and chemotherapy.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following FIG.s.

FIGS. 1A-1H. BCL11B marks an immature cancer cell state associated with chemotherapy resistance in human breast cancer. (A) UMAP representation of the cancer epithelial compartment from 16 human breast tumors (public scRNA-seq dataset available through the Broad Single-Cell portal and GEO: GSE176078; the metaplastic tumor samples were excluded and the IDC tumors were analyzed). (B) UMAP plot displaying the levels of CytoTRACE scores in the cancer epithelial compartment. Higher CytoTRACE scores indicate less differentiated cell states. (C) Plot showing the ranking of genes that are positively associated with CytoTRACE. Only genes with an expression percentage in all cancer cells below the mean percentage minus one standard deviation (<12.42%) were considered, in order to exclude genes with broader expression patterns and identify genes specific to less-differentiated cells. (D) Scatterplot showing gene pairs (double positive)-associated CytoTRACE scores (mean) versus genes (single positive)-associated with CytoTRACE scores (mean). (E) UMAP plots displaying the expression patterns of BCL11B and KRT14 in the cancer epithelial compartment. (F) UMAP plot displaying the expressions of BCL11B and KRT14, as well as the levels of CytoTRACE scores in the cancer epithelial compartment from patient CID4515. (G) Representative images of BCL11B immunohistochemistry staining in human breast cancer samples. (H) Left and middle panel: Kaplan-Meier curve showing differences in relapse-free survival stratified by the median abundance of BCL11B+ cancer epithelial cells in the patients with or without chemotherapy treatment in the METABRIC cohort (n=392, chemotherapy; n=1,503, no chemotherapy); Right panel: Kaplan-Meier curve showing differences in distant recurrence-free survival stratified by the median abundance of BCL11B+ cancer epithelial cells in the Hatzis cohort (n=508).

FIGS. 2A-2M. BCL11B⁺ cells represent a pre-existing drug-resistant cell population. (A) Immunofluorescence co-staining of KRT14/KRT8 in the primary PyMT tumor. (B) Representative immunostaining images of the primary tumor indicate the co-localization of BCL11B and KRT14. (C) Quantifications of the percentage of KRT14⁺/KRT14⁻ cells within the BCL11B⁺ cells and the percentage of BCL11B⁺/BCL11B⁻ cells within the KRT14+ population; n=3 tumors. (D) Upper panel: schematic diagram showing the strategy of the ex vivo drug treatment assay. Lower panel: images showing the expression of tdTomato in the organoids from different groups on day 10. Scale bars, 200 μm (left) and 100 μm (right). (E) Quantification of the percentage of tdTomato⁺ cells in tumor organoids; n=18 organoids from 3 experiments. (F) Single WT or Bcl11b KO tumor cells were cultured in the presence or absence of doxorubicin for 24 hours followed by drug washout. Representative images of tumor organoids on day 11 are shown. Scale bar, 200 μm. (G) Quantification of the frequency of drug-resistant persister organoids (% of control); n=6 cell culture wells from 3 experiments. (H) Control organoids and drug-resistant organoids grown from the WT tumor cells shown in (F) were harvested and analyzed by immunostaining of KRT14 and BCL11B. Scale bar, 50 μm. (I) Percentage of BCL11B⁺ cells in tumor organoids under the indicated conditions; n=10 organoids from 3 experiments. (J) Organoids were pre-grown for 3 days and then treated with doxorubicin (100 nM) for 7 days followed by drug washout. Representative images of tumor organoids are shown. Scale bar, 100 μm. (K) Percentage of actively growing organoids on day 17 in the indicated conditions; n=6 cell culture wells from 3 experiments. (L) Drug-resistant organoids were dissociated into single cells and re-embedded in fresh Matrigel. Immunostaining of KRT14 and BCL11B in the regrown organoids was shown. Scale bar, 50 μm. (M) Tumor growth curves showing WT and Bcl11b KO tumor growth upon saline treatment, or combination treatment (AC, doxorubicin (1 mg/kg)+cyclophosphamide (60 mg/kg)). n=6 mice per group, two-way ANOVA. Nuclei were counter-stained with DAPI (blue). Data are presented as mean±SEM. *p<0.05, **p<0.01, ***p<0.001.

FIGS. 3A-3K. BCL11B functionally governs an immature basal-like cell state with inherent drug-resistance abilities. (A) Volcano plot showing genes differentially expressed between Bcl11b⁺ and Bcl11b⁻ PyMT tumor cells. (B) Heatmap visualization of basal marker genes and myoepithelial marker genes in the bulk RNA-seq dataset of Bcl11b⁺ and Bcl11b tumor cells. (C) KEGG pathway analysis of the upregulated genes in Bcl11b⁺ PyMT tumor cells. (D) Heatmap showing the expression levels of representative drug resistance genes in the bulk RNA-seq data of Bcl11b⁺ and Bcl11b cells. (E) UMAP plots displaying the expression patterns of indicated genes in the scRNA-seq dataset of mammary basal and basal-like cells spanning developmental stages. (F) Left panel: bright-field images of wildtype (WT) and Bcl11b knockout (KO) tumor organoids; Middle panel: immunofluorescence co-staining of KRT14 and BCL11B in WT and Bcl11b KO tumor organoids; Right panel: Immunofluorescence staining of KRT8 in WT and Bcl11b KO tumor organoids. (G) Percentage of KRT14⁺ cells in WT and Bcl11b KO tumor organoids; n=15 organoids from 3 experiments. (H) Real-time PCR analysis of indicated genes in WT and Bcl11b KO tumor cells with Actb as an internal control; n=3 experiments. (I) UMAP representation of 3,377 vehicle-treated tumor organoid cells. The cells are colored by clusters. (J) UMAP plots displaying the expression patterns of indicated genes in the control organoid cells. (K) Boxplot showing the CytoTRACE scores of indicated tumor populations. Nuclei were counter-stained with DAPI (blue). Data are presented as mean±SEM. *p<0.05, **p<0.01, ***p<0.001.

FIGS. 4A-4J Bcl11b⁺ cancer cells demonstrate a notable capability to transcriptionally adapt to drug pressure. (A) UMAP representation of untreated and doxorubicin-treated tumor organoid cells. Cells are colored by treatment conditions. (B) Same UMAP plot as in (A) but colored by cell clusters. (C) The proportions of control and drug-treated cells across cell clusters. (D) UMAP plots showing the expression levels of selected marker genes. (E) Dotplot showing top marker genes for each cluster. (F) Heatmap showing the top 100 marker genes for the Bcl11b^{Hi} basal_Ctrl, Bcl11b^{Hi} basal_Doxo, Bcl11b^{Lo/-} basal_Ctrl, Bcl11b^{Lo/-} basal_Doxo, Lum_Ctrl, and Lum_Doxo. The top pathways enriched in the indicated clusters are shown. (G) Violin plots displaying the expression of representative Cell cycle arrest/Apoptosis genes and Drug Resistance/Survival/GSH metabolism genes across the six subsets. (H) Heatmap showing the expression of indicated drug resistance genes in WT and Bcl11b KO tumor cells treated with vehicle or doxorubicin for 24 hours. (I) UMAP plot showing the levels of CytoTRACE scores in the combined dataset. (J) Boxplot comparing the levels of CytoTRACE scores among the indicated groups.

FIGS. 5A-5H. TNFα enhances chemotherapy efficacy by disrupting the BCL11B⁺ cancer cell state. (A) KEGG pathway analysis of the upregulated genes in Bcl11b⁻ PyMT tumor cells. (B) Left panel: GSEA analysis of the scRNA-seq data demonstrates increased TNFα signaling signature level in Bcl11b⁻ cells compared to Bcl11b⁺ cells. Right panel: GSEA analysis demonstrates increased TNFα signaling signature level in Bcl11b KO cells compared to WT cells. (C) Representative immunostaining images of the control and TNFα-treated tumor organoids. PyMT tumor organoids were treated with TNFα (200 ng/ml) for 6 days and analyzed for BCL11B and KRT14 expression. Scale bar, 50 μm. (D) Percentage of BCL11B⁺ cells or KRT14⁺ cells in tumor organoids with and without TNFα treatment; n=15 organoids from 3 experiments. (E) Tumor organoids were either left untreated or pre-treated with TNFα (200 ng/ml) for 2 days before exposure to doxorubicin (200 nM). After 24-hour drug treatment, single cells from the indicated groups were replated for organoid formation under normal culture conditions. (F) Quantification of the frequency of drug-resistant persister organoids (% of control). n=6 cell culture wells from 3 experiments. (G) Upper panel: Schematic diagram showing the in vivo drug treatment experimental design. NSG mice bearing PyMT tumors were treated with saline, doxorubicin (1 mg/kg, three cycles), TNFα (100 μg/kg, three doses), or doxorubicin in combination with TNFα. Lower panel: Tumor growth curves showing growth of WT or Bcl11b KO tumors under different treatment conditions. n=6 mice per group, two-way ANOVA. (H) Representative images of BCL11B and Ki67 staining in PyMT tumors under different treatments on the indicated days. Scale bar, 50 μm. Nuclei were counter-stained with DAPI (blue). Data are presented as mean±SEM. *p<0.05, **p<0.01, ***p<0.001.

FIGS. 6A-6F. BCL11B promotes chemoresistance in human breast cancer. (A) Violin plots comparing the expression levels of BCL11B, BCL11B signature, KRT14, KRT18, TNF signaling activity, and EPCAM between the BCL11B-high epithelial compartment and BCL11B-low epithelial compartment in the single-cell breast cancer atlas. (B) Real-time PCR quantification of BCL11B mRNA levels in cancer cells isolated from control PDXs and doxorubicin-treated PDXs. GAPDH was used as an internal control. Doxorubicin was administered at three doses (once every 4 days) and tumor cells were collected 1 day after the final dose. n=3 mice per group. (C) Representative images of KRT14 immunostaining in control and drug-treated PDX1 samples. Scale bar, 50 μm. (D) Percentage of KRT14+ cells under the indicated conditions; n=3 tumors. (E) Real-time PCR quantification of BCL11B and KRT14 mRNA levels in control PDX1 and BCL11B knockdown PDX1 cells. n=3 experiments. (F) Left panel: tumor growth curves showing the growth of WT PDX1 tumors or BCL11B knockdown PDX1 tumors upon saline (Vehicle) and doxorubicin (Doxo) treatment. Right panel: tumor growth curves showing the growth of PDX1 tumors upon saline (Vehicle), doxorubicin (Doxo), TNFα, or doxorubicin in combination with TNFα treatments. Treatment schemes are the same as those described in FIG. 5G. n=6 mice per group, two-way ANOVA. Nuclei were counter-stained with DAPI (blue). Data are presented as mean±SEM. *p<0.05, **p<0.01, ***p<0.001.

FIG. 7. BCL11B predetermines a persister state in breast cancer that can be reversed by TNFα treatment. Schematic illustration depicting that BCL11B functionally maintains an immature basal-like tumor population, with BCL11B⁺ cells being in the least differentiated state and possessing a superior capability to transition to a drug-resistant persister state. The BCL11B⁻ basal-like tumor cells retain some level of drug resistance, while luminal tumor cells are drug sensitive and readily undergo cell cycle arrest and cell death upon treatment. TNFα can inhibit BCL11B expression and drive the cells out of the drug-resistant basal-like state, thus significantly improving chemotherapy efficacy.

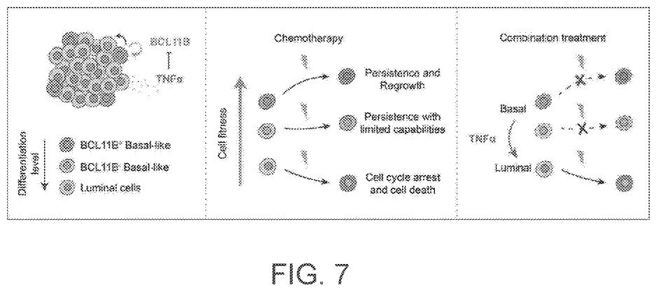

FIGS. 8A-8K. BCL11B represents a distinct marker for less differentiated cancer cells in human breast cancer. (A) UMAP plots showing the cancer epithelial cells colored by tumor subtypes. (B) Violin plots comparing the expression levels of the indicated genes between KRT14⁺ and KRT14⁻ cancer cells. (C) Violin plots comparing the expression level of BCL11B between KRT14⁺ and KRT14⁻ compartments. Seurat clusters with high KRT14 expression were designated as the KRT14⁺ compartment. (D) Violin plots showing the expression levels of the indicated genes across different tumor subtypes. (E) Boxplot comparing the levels of CytoTRACE scores among the indicated groups in the entire cancer compartment. (F) Boxplot comparing the levels of CytoTRACE scores among the indicated groups in the cancer epithelial compartment of patient CID4515. (G) Boxplot comparing the levels of CytoTRACE scores among the indicated groups in the cancer epithelial compartment of patient CID44971. (H) UMAP representation of the cancer epithelial compartment from 30 human breast tumors; pre-treatment samples in cohort 1 were analyzed; the metaplastic tumor samples were excluded from the analysis). (I) Same UMAP plot colored by tumor subtype. (J) Violin plots showing the expression levels of the indicated genes across different tumor subtypes. (K) UMAP plots showing the expression patterns of the indicated genes, as well as the levels of CytoTRACE scores.

FIGS. 9A-9D. BCL11B⁺ cancer cells are associated with chemotherapy resistance in human breast cancer. (A) Quantification of case percentage in each category. High (BCL11B⁺ cells account for >40% of tumor cells), Intermediate (BCL11B⁺ cells account for 20%-40% of tumor cells), and Weak/Negative (BCL11B⁺ cells account for 0-20% of tumor cells). Two breast tissue arrays (BC081116d and BC081116e) were analyzed. (B) Schematic diagram showing the strategy of CIBERSORTx-based tumor cell deconvolution. (C) Boxplots showing the estimated abundances of BCL11B⁺ tumor cells across the breast cancer subtypes in the METABRIC and Hatzis datasets. (D) Upper panel: Kaplan-Meier curve showing differences in relapse-free survival stratified by the median abundance of KRT14⁺/BCL11B⁺ cancer epithelial cells in the METABRIC cohort (n=392, chemotherapy; n=1,503, no chemotherapy) and in the Hatzis cohort (n=508). Lower panel: Same survival analyses as in the upper panel, but based on the abundance of KRT14⁺ population.

FIGS. 10A-10N. BCL11B⁺ tumor cells demonstrate multi-drug resistance ability. (A) Representative FACS plots of tumor cells from PyMT-Bcl11^{btdTomato} mice. Quantification of the percentage of tdTomato+ cells is shown; n=12 tumor samples. (B) Upper panel: A schematic diagram of CRISPR/Cas9-mediated Bcl11b knockout targeting strategy. Lower panel: BCL11B immunostaining confirmed the knockout of Bcl11b in two single clones. (C) Quantification of the frequency of drug-resistant persister organoids in the indicated conditions; n=4 cell culture wells from 3 experiments. (D) Tumor organoid formation in WT or Bcl11b knockout group after paclitaxel treatment (100 or 200 nM). Scale bar, 300 μm. (E) Quantification of the frequency of drug-resistant persister organoids under indicated conditions; n=6 cell culture wells from 3 experiments. (F) Percentage of KRT14⁺ and BCL11B⁺ cells in the regrown organoids; n=15 organoids from 3 experiments. (G) Drug treatment assay in the regrown tumor organoids. Scale bar, 300 μm. (H) Quantification of the frequency of drug-resistant persister organoids from regrown organoid cells under indicated conditions; n=3 experiments. (I) An inducible Bcl11b over-expression vector was introduced to the Bcl11b knockout organoids via lentivirus. Representative images of BCL11B immunostaining in control organoids and doxycycline (Doxy, 100 ng/ml) treated organoids at 48 hours are shown. Scale bar, 50 μm. (J) Bcl11b-KO cells with an inducible Bcl11b overexpressing construct were treated with or without doxycycline for three days and then dissociated into single cells. The single cells were exposed to either doxorubicin or vehicle for 24 hours, followed by drug washout and recovery. Doxycycline was provided to the indicated groups throughout the experiment. Scale bar, 300 μm. (K) Frequency of persister organoids derived from WT or Bcl11b overexpression cells under drug treatment; n=3 experiments. (L) Tumor growth curves showing WT, Bcl11b KO, Bcl11b KO_2 tumor growth upon saline (Ctrl) treatment, or doxorubicin treatment (1 mg/kg). n=5 mice per group, two-way ANOVA. (M) Representative images of KRT14 and BCL11B immunostaining in untreated or AC (doxorubicin+cyclophosphamide)-treated PyMT tumor samples (2 days or 6 days after the final treatment). (N) Percentage of KRT14+ and BCL11B⁺ cells in WT tumor under indicated conditions; n=6 tumors. Nuclei were counter-stained with DAPI (blue). Data are presented as mean±SEM. Scale bar, 50 μm. *p<0.05, **p<0.01, ***p<0.001.

FIGS. 11A-11F. The Bcl11b⁺ cancer cell population is linked to a rudimentary basal-like cell state during development with an intrinsic drug resistance program. (A) UMAP plot of Krt14-positive basal and basal-like cells across 4 different developmental stages of the mammary gland (GEO: GSE164017). The Krt14-positive cell clusters from the different stages were combined and analyzed together. (B) Violin plots showing the expression score of the top 50 Bcl11b co-expressed genes in tumors within the basal and basal-like mammary cells across developmental stages. (C) UMAP plots displaying the expression patterns of indicated genes in the scRNA-seq dataset of mammary basal and basal-like cells. (D) Immunofluorescence staining of orthotopic tumors derived from WT or Bcl11b knockout organoid cells. Nuclei were counter-stained with DAPI (blue). Scale bar, 50 μm. (E) Percentage of KRT14+ cells in WT and Bcl11b KO tumors; n=6 tumors. (F) Expression levels of the basal cell markers, luminal cell markers, and selected drug resistance genes in the bulk RNA-seq dataset of Bcl11b WT and knockout cells. Data are presented as mean±SEM. *p<0.05, **p<0.01, ***p<0.001.

FIGS. 12A-12D. Single-cell RNA-seq analysis of treatment-naïve tumor organoids. (A). Schematic diagram showing the scRNA-seq experimental design. (B) Quantifications of the percentage of BCL11B^{Hi}, BCL11B^{Lo} and BCL11B⁻ cells within the KRT14⁺ cell population in tumor organoids; n=7 tumor organoids. Data are presented as mean±SEM. The representative immunostaining image of tumor organoids is shown on the left. KRT14, green; BCL11B, purple. Nuclei were counter-stained with DAPI (blue). Scale bar, 50 μm. (C) UMAP plots showing the expression levels of selected marker genes in the vehicle-treated control tumor cells. (D) Heatmap showing top marker genes for each cluster in the control dataset.

FIGS. 13A-13E. Bcl11b⁺ tumor cells maintain the differentiation potential and display a higher tumorigenic ability. (A) Representative images of Bcl11^{btdTomato} immunofluorescence, KRT14, and KRT8 staining in tumor organoids grown from FACS-sorted single Bcl11^{btdTomato} positive cells. Scale bar, 50 μm. (B) Representative images of tumor organoids generated from the indicated number of Bcl11^{btdTomato} positive or Bcl11^{btdTomato} negative tumor cells. (C) Quantification of the number of organoids generated from the indicated number of cells; n=3 experiments. (D) Photographs of representative tumors from mice engrafted with the indicated number of Bcl11^{btdTomato} positive or Bcl11^{btdTomato} negative tumor cells. (E) Comparison of BCL11B expression in non-tumorigenic and tumorigenic samples of human breast tumors (GEO: GSE6883). Nuclei were counter-stained with DAPI (blue). Data are presented as mean±SEM; n=3. *p<0.05, **p<0.01, ***p<0.001.

FIGS. 14A-14D. Bcl11b⁺ basal-like cells are tolerant to chemotherapy treatment. (A) UMAP plots showing the expression levels of cell cycle arrest and apoptosis-associated genes in the combined dataset. (B) Violin plots showing the changes in Mki67 expression in the basal and luminal compartments upon the drug treatment. (C) Relative proportions of Mki67+ basal and luminal tumor cells in the untreated and drug-treated tumor organoids. (D) Violin plots displaying the expression of various drug resistance genes across the six subsets.

FIGS. 15A-15D. BCL11B counteracts the anti-tumor effects exerted by doxorubicin. (A) Cell cycle analysis of WT and Bcl11b KO tumor organoids treated with vehicle, 100 nM and 200 nM doxorubicin for 24 hours. Representative flow plots and quantification of the proportion of cells in each cell cycle phase are shown. (B) Representative γH2AX staining in the PyMT tumor at 48 hours after treatment with vehicle or chemotherapeutic drugs (doxorubicin+cyclophosphamide). Scale bar, 50 μm. (C) WT or Bcl11b-deficient tumor organoids were treated with doxorubicin (100 nM) for 3 hours and 24 hours. Representative images of BCL11B immunostaining and doxorubicin fluorescence at different time points are shown. Doxorubicin autofluorescence can be used to measure the intracellular drug concentration. Scale bar, 50 μm. (D) The fluorescence intensity of doxorubicin was quantified in BCL11B and BCL11B⁺ cells after 3, 24 or 48-hour treatment; n=15 organoids from 3 experiments. Nuclei were counter-stained with DAPI (blue). Data are presented as mean±SEM. *p<0.05, **p<0.01, ***p<0.001.

FIGS. 16A-16C. Bcl11b⁺ basal-like cells are distinct from cells with EMT activation. (A) Violin plots displaying the expression of indicated marker genes across the six subsets. (B) UMAP plots showing the expression levels of EMT-related genes in the combined dataset. (C) Enrichment analysis of the Normal luminal signature, Normal basal signature, and EMT signature (dbEMT gene set) in indicated subsets of the tumor cells.

FIGS. 17A-17G. TNFα treatment prevents the emergence of cancer persisters by disrupting BCL11B expression. (A) Upper panel: heatmap displaying the expression pattern of selected genes in Bcl11b⁺ and Bcl11b⁻ cells in the scRNA-seq dataset of normal mouse mammary epithelial cells (GEO: GSE109774). Lower panel: heatmap displaying the expression pattern of selected genes in Bcl11b⁺ and Bcl11b⁻ cells in the single-cell RNA-seq data of PyMT organoids. (B) Western blot analysis of BCL11B expression upon treatment of TNFα. (C) Overexpression of BCL11B inhibits TNFα-induced ERK phosphorylation. WB analysis of the level of p-ERK1/2 upon TNFα treatment in the absence or presence of doxycycline is shown. (D) Immunofluorescence analysis of KRT14, KRT8, and Bcl11b-tdTomato in the tumor organoids derived from single Bcl11^{btdTomato} positive cells in the absence or presence of TNFα treatment. Scale bar, 50 μm. (E) Quantification of the percentage of KRT14⁺ cells in the tumor organoids derived from single Bcl11^{btdTomato} positive cells under different conditions; n=10 organoids from 3 experiments. (F) Quantification of the percentage of Ki67⁺ cells in indicated groups; n=3 tumors. (G) Quantification of the percentage of BCL11B⁺ cells in indicated groups; n=3 tumors. Data are presented as mean±SEM. *p<0.05, **p<0.01, ***p<0.001.

FIGS. 18A-18E. The BCL11B⁺ cell state is conserved in human breast cancer. (A) UMAP plots showing the cancer epithelial cells in the single-cell atlas colored by BCL11B-high and BCL11B-low groups. (B) Schematic diagram showing the strategy of generating the ‘BCL11B signature’ based on bulk RNA-seq analysis. (C) Correlation between Bcl11b and the BCL11B signature in the scRNA-seq data of tumor organoids. (D) Violet plots showing the expression of selected genes in the BCL11B-high and BCL11B-low compartments. (E) UMAP plots displaying the expression patterns of EPCAM, the myoepithelial signature (ACTA2, ACTG2, MYH11, MYL9, TAGLN, and MYLK), SNAI1, and ZEB1 in the epithelial cell compartment of human breast cancer.

FIGS. 19A-19E. BCL11B promotes treatment resistance in human breast cancer. (A) Real-time PCR quantification of BCL11B and KRT14 mRNA levels in control PDX2 and BCL11B knockdown PDX2 cells. n=3 experiments. (B) Tumor growth curves showing the growth of WT PDX2 tumors or BCL11B knockdown PDX2 tumors upon saline (Vehicle) and doxorubicin (Doxo) treatment. n=6 mice per group, two-way ANOVA. (C) BCL11B expression in breast cancer cell lines versus drug sensitivity AUC for chemotherapeutic drugs based on the data from the DepMap and GDSC databases. A higher AUC value implies less drug sensitivity. (D) Drug treatment assay in control MCF10A cells and BCL11B over-expressing MCF10A cells. MCF10A cells with an inducible BCL11B overexpressing construct were treated with or without doxycycline for three days and then dissociated into single cells. The single cells were cultured in Matrigel in the presence or absence of doxorubicin for 24 hours, followed by drug washout and recovery. Doxycycline was provided to the indicated groups for the duration of the experiment. Scale bar, 300 μm. (E) Quantification of the frequency of drug-resistant organoids in the indicated conditions; n=6 cell culture wells from 3 experiments. Data are presented as mean±SEM. *p<0.05, **p<0.01, ***p<0.001.

FIG. 20. Graph of BCL11B expression versus relapse-free survival. Utilizing a threshold of 10% or 20% effectively separates the patients.

### DETAILED DESCRIPTION

Before the present methods and compositions are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

Breast Cancer. Most breast cancers are epithelial tumors (carcinomas) that develop from cells lining ducts or lobules; nonepithelial cancers of the supporting stroma (eg, angiosarcoma, primary stromal sarcomas, phyllodes tumor) are less common. Epithelial cancers are divided into carcinoma in situ and invasive cancer.

Carcinoma in situ is proliferation of cancer cells within ducts or lobules but without invasion of stromal tissue. There are 2 types: Ductal carcinoma in situ (DCIS): DCIS refers to neoplastic epithelial lesions that are confined to the breast ducts; lesions may have varying histology and malignant potential. Lobular carcinoma in situ (LCIS): LCIS is often multifocal and is bilateral in approximately 20 to 60% of cases. Classic LCIS is not malignant but increases the risk of developing invasive carcinoma in either breast.

Invasive carcinoma is primarily adenocarcinoma. Epithelial cancers may express hormone receptors. Approximately 80% of postmenopausal and 20% of premenopausal patients with breast cancer have an estrogen receptor-positive (ER+) tumor; approximately 70% of all breast cancers are progesterone receptor-positive. Another cellular receptor is human epidermal growth factor receptor 2 (HER2; also called HER2/neu or ErbB2); its presence correlates with a poorer prognosis at any given stage of cancer. The majority of breast cancers are hormone receptor-positive and HER2 negative (approximately 70%); 12% are triple negative (hormone receptor-negative and HER2-negative).

Staging follows the TNM (tumor, node, metastasis) classification (see table Anatomic Staging of Breast Cancer). Because clinical examination and imaging have poor sensitivity for nodal involvement, staging is refined during surgery, when regional lymph nodes can be evaluated.

Endocrine therapy, chemotherapy, or HER2-directed therapy delays or prevents recurrence in almost all patients and prolongs survival in some. Decisions about the type of adjuvant therapy are made based on tumor characteristics, including estrogen receptor (ER) and progesterone receptor (PR) status, presence of human epidermal growth factor 2 (HER2) protein, grade and stage (including lymph node involvement), and genomic risk stratification. Some patients are treated with a combination of endocrine therapy and chemotherapy. Chemotherapy is usually begun soon after surgery. If systemic chemotherapy is not required, endocrine therapy is usually begun soon after surgery and is continued for 5 to 10 years.

Medications used for endocrine therapy include tamoxifen and aromatase inhibitors. Tamoxifen is a selective estrogen receptor modulator that competitively binds with estrogen receptors. Aromatase inhibitors (anastrozole, exemestane, letrozole) block peripheral production of estrogen in postmenopausal women.

Combination chemotherapy regimens are more effective than a single medication. Dose-dense regimens given for 4 to 6 months are preferred; in dose-dense regimens, the time between doses is shorter than that in standard-dose regimens. There are many regimens; a commonly used one is ACT (doxorubicin plus cyclophosphamide followed by paclitaxel). The most effective chemotherapy agents are capecitabine, doxorubicin (including its liposomal formulation), gemcitabine, the taxanes paclitaxel and docetaxel, and vinorelbine. Response rate to a combination of agents is higher than that to a single agent, but survival is not improved and toxicity is increased. Thus, some oncologists use single agents sequentially.

If the tumor overexpresses HER2 (HER2+), anti-HER2 monoclonal antibodies (trastuzumab, pertuzumab) may be used. Adding the humanized monoclonal antibody trastuzumab to chemotherapy provides substantial benefit.

B cell leukemia 11b (Bcl11b) is a zinc finger protein transcription factor with a multiplicity of functions. It works as both a genetic suppressor and activator, acting directly, attaching to promoter regions, as well as indirectly, attaching to promoter-bound transcription factors. See, for example, NCBI Reference Sequence: NG_027894.1. BCL11b is a Kruppel-like C₂H₂ zinc finger protein transcription factor. The encoding gene, present in human chromosome 14, was initially found to be a tumor suppressor gene closely related to Bcl11a, a proto-oncogene. Bcl11b has alpha and beta splice isoforms which comprise 823 (89.1 kDa) and 894 (95.5 kDa) amino acids respectively in humans. The beta isoform includes Exon 3 whereas this is spliced out in the alpha but both contain Exon 4 which includes the six critical C₂H₂ zinc finger binding domains and thus are functionally similar. Of those, domains 3, and 4 bind to the DNA, while other regions are important for protein interactions. The murine Bcl11b gene is 88% similar to the human gene and is on chromosome 12 rather than 14. Bcl11b acts both directly, by attaching to promoter regions, and indirectly, by binding to transcription factors bound to promoter regions themselves. In its role as a transcription factor Bcl11b acts as both a repressor and activator of a multiplicity of genes.

TNFα. Tumor necrosis factor (TNF). The human TNF gene is mapped to chromosome 6p21.3, residing in the class III region of the major histocompatibility complex, NCBI Reference Sequence: NG_007462.1. The transcribed region contains 4 exons separated by 3 introns, for a total of 2,762 base pairs in the primary transcript and 1,669 base pairs in the mRNA. The mRNA consists of four regions: the 5′ untranslated region, which is not included in the TNF protein; the transmembrane portion, which is present in transmembrane TNF but not in soluble TNF; the soluble portion; and the 3′ untranslated region.

TNF is initially produced as a transmembrane protein (tmTNF) consisting of 233 amino acids. tmTNF binds to both TNFR1 and TNER2, but its activity is primarily mediated by TNFR2. Upon binding to a receptor, tmTNF also activates signaling pathways within its own cell. tmTNF is cleaved by TNF alpha converting enzyme (TACE), which causes the extracellular portion to be secreted. After cleavage, the remaining tmTNF is cleaved again by SPPL2B, causing the intracellular portion to translocate to the nucleus. There, it is believed to regulate cytokine production, such as triggering the expression of interleukin-12.

The secreted extracellular portion, denoted sTNF, consists of 157 amino acids. Unlike tmTNF, sTNF can only bind to TNFR1. The secondary structure of sTNF consists primarily of alternating strands that join into two sheets, known as antiparallel β-sheets. The two sheets are layered on top of each other, forming a wedge shape known as an antiparallel β-sandwich. The last 9 residues of the C-terminus are locked into the middle strand of the bottom sheet, and are necessary for bioactivity. Both tmTNF and sTNF are only bioactive as homotrimers, whereas individual monomers are inactive.

In some cancers, TNF has been shown to play an inhibitory role, primarily when injected locally, repeatedly, and at high concentrations. Due to TNF's adverse side effects, potential TNF cancer treatments seek to maximize cytotoxicity to tumors while minimizing exposure to the entire body. Some treatments increase cytotoxicity by inhibiting the cell survival pathways of tumors before treatment with TNF. Other treatments localize TNF activity using antibody-TNF fusions, also known as immunocytokines.

Agonists of TNF mimic or enhance the activity of the protein. Agonists of TNFα include, for example, recombinant human TNFα (rTNFα), where pegylated or modified TNFα variants may be used to improve pharmacokinetics and reduce systemic toxicity. Agonistic antibodies against TNFR1 or TNFR2 can bind and activate one or both of TNFR1 and TNFR2, selectively triggering apoptosis (via TNFR1) or immune cell activation (via TNFR2). Oncolytic viruses encoding TNFα that are engineered to express TNFα within tumors have also been described.

Agonists may be locally delivered to a tumor site, e.g. using nanoparticle delivery, localized injections, or engineered viruses.

The effective dose of a TNFα agonist, e.g. recombinant human TNFα, may be from about 0.1 mg to about 10 mg/dose, from about 0.5 to about 5 mg/dose, from about 1 to about 4 mg, and may be around 0.1 mg, 0.5 mg, 1 mg, 2.5 mg, 5 mg, 10 mg. The dosage may be appropriately adjusted for other agents.

TNFα is generally administered in combination with suitable chemotherapeutic agents as disclosed herein, including with limitation a regimen comprising administration of an anthracycline agent, e.g. doxorubicin (adriamycin), daunorubicin, epirubicin, idarubicin, valrubicin, mitoxantrone, etc.

“In combination with”, “combination therapy” and “combination products” refer, in certain embodiments, to the concurrent administration to a patient of a first therapeutic (i.e., first therapeutic agent) and a second therapeutic agent. When administered in combination, each component can be administered at the same time or sequentially in any order at different points in time. Thus, each component can be administered separately but sufficiently closely in time so as to provide the desired therapeutic effect.

“Concomitant administration” of a first and second therapeutic agent means administration of the first agent and second agent at such time that both the first agent and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

Chemotherapy may include administration of chemotherapeutic agents such as: Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinoIAQ (Cisplatin), PlatinoIAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting PD-L1 and those targeting CTLA-4 such as ipilimumab.

Antibiotics, e.g. antibiotics with the classes of aminoglycosides; carbapenems; and the like; penicillins, e.g. penicillin G, penicillin V, methicillin, oxacillin, carbenicillin, nafcillin, ampicillin, etc. penicillins in combination with β-lactamase inhibitors, cephalosporins, e.g. cefaclor, cefazolin, cefuroxime, moxalactam, etc; tetracyclines; cephalosporins; quinolones; lincomycins; macrolides; sulfonamides; glycopeptides including the anti-infective antibiotics vancomycin, teicoplanin, telavancin, ramoplanin and decaplanin. Derivatives of vancomycin include, for example, oritavancin and dalbavancin (both lipoglycopeptides). Telavancin is a semi-synthetic lipoglycopeptide derivative of vancomycin (approved by FDA in 2009). Other vancomycin analogs are disclosed, for example, in WO 2015022335 A1 and Chen et al. (2003) PNAS 100(10): 5658-5663, each herein specifically incorporated by reference. Non-limiting examples of antibiotics include vancomycin, linezolid, azithromycin, daptomycin, colistin, eperezolid, fusidic acid, rifampicin, tetracyclin, fidaxomicin, clindamycin, lincomycin, rifalazil, and clarithromycin.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

The agents may be administered one or a plurality of days, and in some embodiments is administered daily, every two days, semi-weekly, weekly, etc. for a period of from about 1, about 2, about 3, about 4, about 5, about 6, about 7 or more weeks, up to a chronic maintenance level of dosing. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

Treating may refer to any indicia of success in the treatment or amelioration or prevention of disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with *****. The term “therapeutic effect” refers to the reduction, elimination, or prevention of the disease, symptoms of the disease, or side effects of the disease in the subject.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indicated and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

Determining a therapeutically or prophylactically effective amount of therapeutic agents or compositions of agents can be done based on animal data using routine computational methods. In one embodiment, the therapeutically or prophylactically effective amount contains between about 0.01 mg and about 1 g of an agent, e.g. nucleic acid, protein or peptide, etc., as applicable. In another embodiment, the effective amount contains between about 1 mg and about 100 mg of agent, as applicable.

Administering therapeutic agents can be effected or performed using any of the various methods and delivery systems known to those skilled in the art. The administering can be performed, for example, intravenously, orally, via implant, transmucosally, transdermally, intramuscularly, intrathecally, and subcutaneously. The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone. Nucleic acids of the invention can also be administered attached to particles using a gene gun.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and Jun. 2, 2005 antioxidants (e.g., parabens, vitamins E and C, and ascorbic acid), anti-caking agents, coating agents, and chelating agents (e.g., EDTA).

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having a disease of interest. Subjects may be human, but also include other mammals, particularly those mammals useful as laboratory models for human disease, e.g. mouse, rat, etc.

The definition of an appropriate patient sample encompasses solid tissue samples such as a biopsy specimen or tissue cultures or cells derived there from and the progeny thereof. The definition also includes samples that have been manipulated in any way after their procurement, such as by treatment with reagents; washed; or enrichment for certain cell populations, such as endometrial cells, etc. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample comprising polynucleotides and/or polypeptides that is obtained from a patient's sample cell (e.g., a cell lysate or other cell extract comprising polynucleotides and/or polypeptides); and a sample comprising sample cells from a patient. A biological sample comprising a sample cell from a patient can also include normal, non-diseased cells.

Samples can be obtained once or multiple times from an individual. Multiple samples can be obtained from different locations in the individual (e.g., biopsy samples), at different times from the individual, or any combination thereof.

When samples are obtained as a series, the samples can be obtained at fixed intervals, at intervals determined by the status of the most recent sample or samples or by other characteristics of the individual, or some combination thereof. It will be appreciated that an interval may not be exact, according to an individual's availability for sampling and the availability of sampling facilities, thus approximate intervals corresponding to an intended interval scheme are encompassed by the invention.

When necessary, cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells can be fixed, e.g. with 3% paraformaldehyde, and are usually permeabilized, e.g. with ice cold methanol; HEPES-buffered PBS containing 0.1% saponin, 3% BSA; covering for 2 min in acetone at −200C; and the like as known in the art and according to the methods described herein.

“Measuring” or “measurement” in the context of the present teachings refers to determining the presence, absence, quantity, amount, or effective amount of BCL11B-expressing cells in a clinical or subject-derived sample, including the presence, absence, or concentration levels of such substances, and/or evaluating the values or categorization of a subject's clinical parameters based on a control, e.g. baseline levels of the marker.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In some embodiments measurement is performed by quantitative PCR (qPCR, also referred to as real time-PCR and digital PCR (dPCR). qPCR, also known as real-time PCR, quantifies mRNA indirectly by converting it into complementary DNA (cDNA) using reverse transcriptase and amplifying specific sequences with fluorescent dyes or probes. The fluorescence emitted during each cycle correlates with the amount of amplified DNA, providing real-time data on mRNA abundance. This method is highly sensitive and widely used for gene expression studies. dPCR, on the other hand, partitions the cDNA into thousands of individual reactions before amplification, enabling absolute quantification without the need for standard curves. It counts the number of positive partitions containing the target sequence, making it ideal for detecting low-abundance mRNAs or rare transcripts. Both methods can be coupled with high-throughput technologies, such as microfluidic-based systems, for analyzing multiple mRNA targets simultaneously, enhancing their application in transcriptomics and molecular diagnostics.

In other embodiments a cell population is contacted with an “affinity reagent”, or “specific binding member”, such as an antibody, ligand, etc. that selectively binds to a protein or marker of the invention. The term “affinity reagent” includes any molecule, e.g., peptide, nucleic acid, small organic molecule. For some purposes, an affinity reagent selectively binds to a cell surface marker. For other purposes an affinity reagent selectively binds to a cellular signaling protein.

In some embodiments, the affinity reagent is a peptide, polypeptide, oligopeptide or a protein, particularly antibodies and specific binding fragments and variants thereof. The peptide, polypeptide, oligopeptide or protein can be made up of naturally occurring amino acids and peptide bonds, or synthetic peptidomimetic structures. Thus “amino acid”, or “peptide residue”, as used herein include both naturally occurring and synthetic amino acids. Proteins including non-naturally occurring amino acids can be synthesized or in some cases, made recombinantly.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and possess other variations.

The methods the invention may utilize affinity reagents comprising a label, labeling element, or tag. By label or labeling element is meant a molecule that can be directly (i.e., a primary label) or indirectly (i.e., a secondary label) detected; for example a label can be visualized and/or measured or otherwise identified so that its presence or absence can be known.

In one embodiment of the invention, the methods of determining propensity to develop resistance to chemotherapy in breast cancer comprise obtaining a patient sample comprising breast cancer cells, e.g. a biopsy sample. The sample may be obtained prior to treatment, e.g. prior to radiation, prior to chemotherapy. The sample may be obtained following treatment, e.g. in a follow-up biopsy. The breast cancer cells, or biomolecules derived therefrom may be physically contacted with an affinity reagent specific for BCL11B. In some embodiments the affinity reagent is an antibody specific for BCL11B. Suitable methods of analysis include, without limitation, flow cytometry, mass cytometry, confocal microscopy, and the like. In other embodiments the sample is subjected to RNA analysis, e.g. RT-PCR, qPCR, RT-qPCR, RNA sequencing, etc. The number of cancer cells positive for expression of BCL11B is determined. The data is compared to measurements from a baseline cell population, e.g. a normal control, non-tumor sample from the individual, etc. The data can be normalized for comparison.

In an embodiment, the method further comprises selecting a treatment regimen for the patient based on the analysis. Treatment regimens of interest can include decision-making for treating the patient with a combination of an effective dose of TNFα, and chemotherapy.

In other embodiments of the invention a device or kit is provided for the analysis of patient samples. Such devices or kits will include reagents that specifically identify expression of BCL11B, including without limitation affinity reagents, PCR primers, etc. The reagents can be provided in isolated form, or pre-mixed with reagents suitable for the methods of the invention. A kit can include instructions for using the reagents to determine data from the sample; and instructions for statistically analyzing the data. Such a system may include a software component configured for analysis of data obtained by the methods of the invention.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

BCL11B Predetermines a Persister State in Breast Cancer that is Reversed by TNFα Treatment

The frequent development of chemoresistance in cancer presents a significant clinical challenge, yet the underlying causes for heterogeneous drug responses remain largely elusive. Here, we systematically assessed the cell differentiation status of human breast cancer cells utilizing single-cell atlases and identified a distinct population of immature basal-like cancer cells marked by BCL11B. Notably, higher levels of BCL11B⁺ cancer cells are significantly associated with early relapse in breast cancer patients who received chemotherapy. Integrating human and mouse breast cancer models and functional assays, we uncovered that BCL11B⁺ cancer cells can preferentially transition to a drug-resistant persister state during treatment, while BCL11B⁻ luminal tumor cells are drug-sensitive and readily undergo cell cycle arrest and apoptosis. Functioning as a central regulator, BCL11B delineates a pre-existing immature cell state that not only possesses an intrinsic drug-resistance program but also demonstrates a remarkable capacity to adapt transcriptionally upon treatment. Importantly, the cytokine tumor necrosis factor alpha (TNFα) is revealed as a natural inhibitor of BCL11B and can directly reverse the emergence of chemoresistant persisters. Therefore, we identify BCL11B as a pre-determinant of drug response, where a TNFα combination treatment can be used for a subset of breast cancer patients at high risk of developing chemoresistance.

We identified that a subset of human breast cancers harbors a distinct population of epithelial BCL11B⁺ tumor cells, resulting in a higher risk of chemoresistance. Combining patient data, human breast cancer models, and the mouse MMTV-PyMT tumor model, we identified that this pre-existing tumor subpopulation is in a unique immature basal cell state that is governed by Bcl11b, a previously established stem cell marker in the mouse mammary gland. Through single-cell profiling, we unraveled a previously undescribed drug response diversity among tumor subpopulations. The Bcl11b⁺ basal tumor subpopulation can preferentially survive the treatment and transcriptionally adapt to the drug pressure. By contrast, the luminal tumor cells are drug-sensitive and readily undergo cell cycle arrest and apoptosis upon treatment. Importantly, tumor necrosis factor alpha (TNF-α) treatment can effectively target this basal cell state and thus remarkably increase chemotherapy efficacy in tumors with high levels of BCL11B. Therefore, we identify BCL11B as a biomarker and an actionable therapeutic target for combating chemoresistance in breast cancer.

### Results

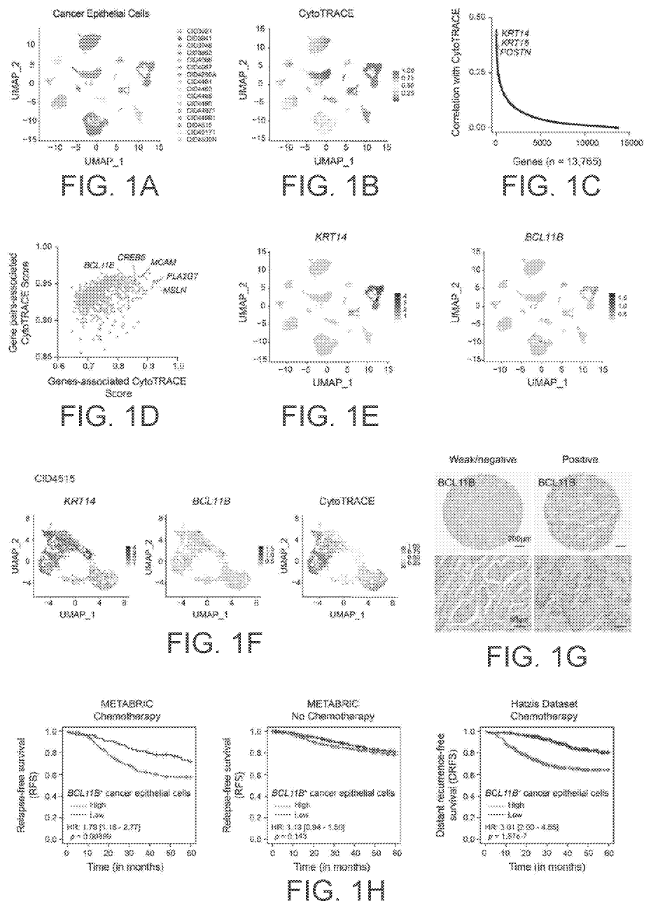

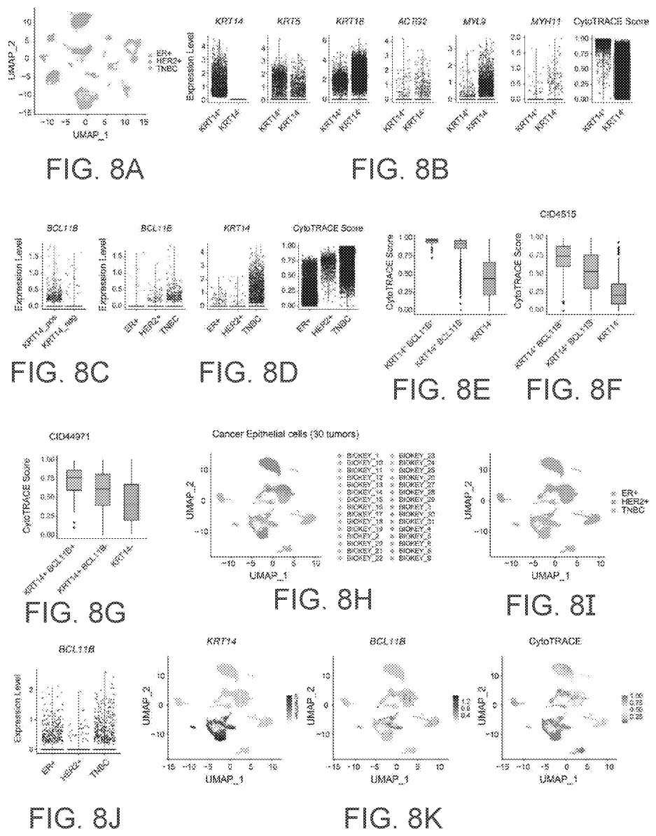

BCL11B marks a distinct immature cancer cell population in human breast cancer. We have previously shown that the CytoTRACE model, a robust computational framework for predicting cellular differentiation states in scRNA-seq datasets, can be used to identify both normal stem cells and immature cells within cancers. To systematically investigate the immature cell states in breast cancer and reveal genes associated with tumor characteristics including therapy resistance, we applied CytoTRACE analysis to the whole cancer epithelial compartment in a single-cell atlas encompassing ER+, HER2+, and triple-negative breast cancer (TNBC) subtypes. Cancer cells from different patient samples were mainly grouped into separate clusters and exhibited varied levels of differentiation as inferred by CytoTRACE (FIG. 1, A and B, and FIG. 8A), aligning with the concept of inter-tumor heterogeneity. The TNBC cells generally exhibited lower levels of differentiation compared to other cancer cells, while within each tumor sample, including TNBC, there were usually tumor subpopulations in a relatively less differentiated state (FIG. 1B).

To explore common markers for the less differentiated cancer cells across the dataset, we ranked genes based on their correlation with CytoTRACE. Interestingly, KRT14 was identified as one of the top three genes associated with less differentiated cells, along with other basal genes, such as KRT16 and POSTN, ranking at the top of the list (FIG. 1C). While KRT14 is typically seen as a marker for basal cells, it is also found in cells with basal-luminal intermediate features in the normal human breast. Here, KRT14⁺ cancer cells exhibited low levels of myo-contractile genes expressed by mature basal cells (FIG. 8B), indicating an immature basal-like cell state distinct from the differentiated phenotype. Upon further examination of the KRT14⁺ populations, we observed that there were subsets of cells with low CytoTRACE scores (FIG. 8B). To distinguish the least differentiated from the more mature KRT14⁺ populations, we paired each of the top 1,000 genes identified by CytoTRACE with KRT14 and examined the mean CytoTRACE scores associated with each gene and gene pair. This analysis yielded a list of candidate genes that not only predict less differentiated cells independently in the whole dataset, but also mark subsets of KRT14⁺ cells in the least differentiated state (FIG. 1D). Remarkably, BCL11B, a marker gene for a minority quiescent stem cell population that we previously identified in the mouse mammary epithelium, emerged as one of the top 30 candidate genes (FIG. 1D). Many of the other top candidate genes have been previously linked to increased tumorigenic ability and invasiveness in breast cancer, such as PLA2G7, MSLN, MCAM, and CREB5 (FIG. 1D), validating the effectiveness of our approach.

We next focused on BCL11B and found that BCL11B⁺ cells represented distinct tumor subpopulations in a subset of breast tumors and were primarily located in the KRT14⁺ compartments. These cells were present in all three major cancer subtypes, with a higher frequency observed in TNBC (FIG. 1E and FIG. 8, C and D). The BCL11B⁺ cells showed a strong correlation with less differentiated cancer cells across the dataset, with the KRT14⁺/BCL11B⁺ cells exhibiting the highest CytoTRACE scores compared to other tumor subsets (FIG. 1E and FIG. 8E). As CytoTRACE outputs the relative ordering of all cells analyzed in the dataset, we further studied individual tumor samples and confirmed that BCL11B marked subpopulations of KRT14⁺ cells in the more immature state (FIG. 1F and FIG. 8, F and G). To validate our findings, we examined a separate single-cell atlas of human breast cancers, which consisted of 12 TNBC, 15 ER+, and 3 HER2+ tumors (FIG. 8, H and I). Consistently, BCL11B marked tumor subpopulations in a subset of patient samples spanning all three cancer subtypes and was more specifically enriched in cancer cells in the more immature states (FIG. 8, J and K).

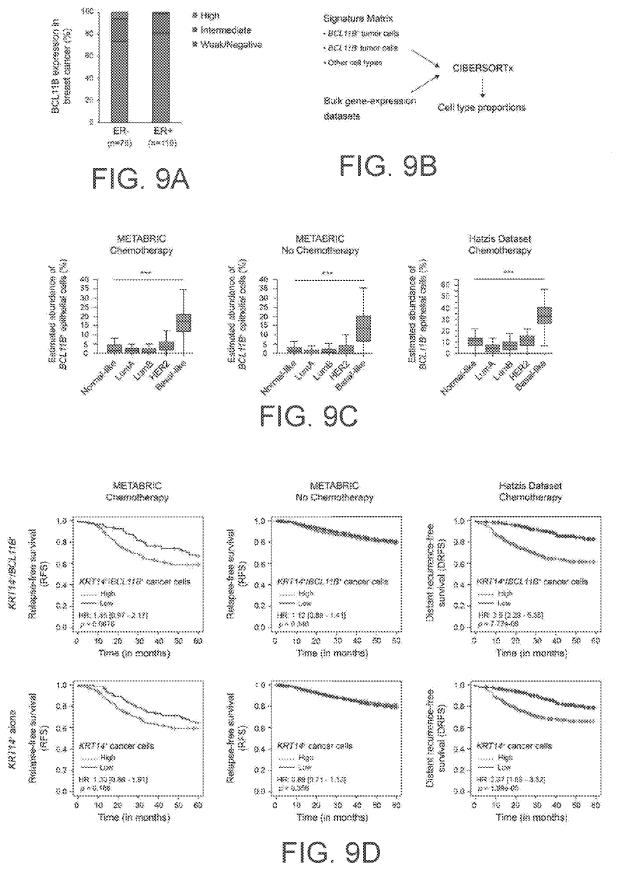

To examine the protein expression pattern of BCL11B in cancer tissues, we conducted tissue microarray analysis of 188 breast tumor samples and observed a similar inter-tumor heterogeneity. We found prominent BCL11B expression in approximately 27% of ER-negative tumors and 19% of ER-positive tumors, with the highest expression levels mainly observed in the ER-negative subtype (FIG. 1G and FIG. 9A). In addition, in the majority of BCL11B positive cases, only a subset of tumor cells, typically located at the tumor-stromal border, displayed high expression of BCL11B (FIG. 1G and FIG. 9A). Therefore, our analysis combining different single-cell atlas datasets and tissue microarray assessments revealed that BCL11B⁺ cells represent a distinct tumor subpopulation in a group of breast cancer patients and exhibit markedly immature cell features.

BCL11B⁺ cancer cells are associated with chemotherapy resistance in breast cancer. Next, we sought to understand the clinical significance of BCL11B expression in breast cancer. To minimize the influence of non-cancerous expression of BCL11B in tumors, we utilized a deconvolution-based approach to focus on the BCL11B-expressing cancer cells in bulk gene expression datasets. We defined transcriptional signatures for BCL11B⁺ cancer cells, BCL11B⁻ cancer cells, and various niche cell types based on the single-cell atlas, and used CIBERSORTx to estimate the abundance of each cell population in the METABRIC cohort (FIG. 9B). Notably, we observed a strong association between higher levels of BCL11B⁺ cancer cells and early relapse (within 5 years) specifically in breast cancer patients who had received chemotherapy (FIG. 1H). No such correlation was found when chemotherapy was not given (FIG. 1H). The estimated levels of BCL11B⁺ tumor cells in breast cancer subtypes were comparable between the chemotherapy-treated and non-treated patients, indicating the robustness of the deconvolution method (FIG. 9C). To validate this finding, we further analyzed the Hatzis breast cancer dataset (33), where over 93% of the patients received neoadjuvant chemotherapy. Consistently, higher abundances of BCL11B⁺ tumor cells were found to have a significant correlation with early distant recurrence in this chemotherapy-treated cohort (FIG. 1H).

To explore if KRT14⁺/BCL11B⁺ would perform better than BCL11B alone in predicting patient response, we defined the transcriptional signature for cancer cells co-expressing both genes in the scRNA-seq data and then performed deconvolution analysis. Despite the technical challenges in accurately identifying double-positive cells in scRNA-seq datasets, it is evident that higher levels of KRT14⁺/BCL11B⁺ cells are linked to a poor prognosis in both chemotherapy-treated cohorts (FIG. 9D). However, the potential of KRT14⁺/BCL11B⁺ as a more effective marker than BCL11B alone could not be established, likely due to technical constraints. In contrast, BCL11B alone outperformed KRT14 alone in predicting the poor prognosis in response to chemotherapy, consistent with the least differentiated state of BCL11B⁺ cells within the KRT14⁺ population (FIG. 9D). Together, these data demonstrated a specific association between the elevated levels of BCL11B⁺ cancer cells and the high risk of developing chemotherapy resistance in human breast cancer.

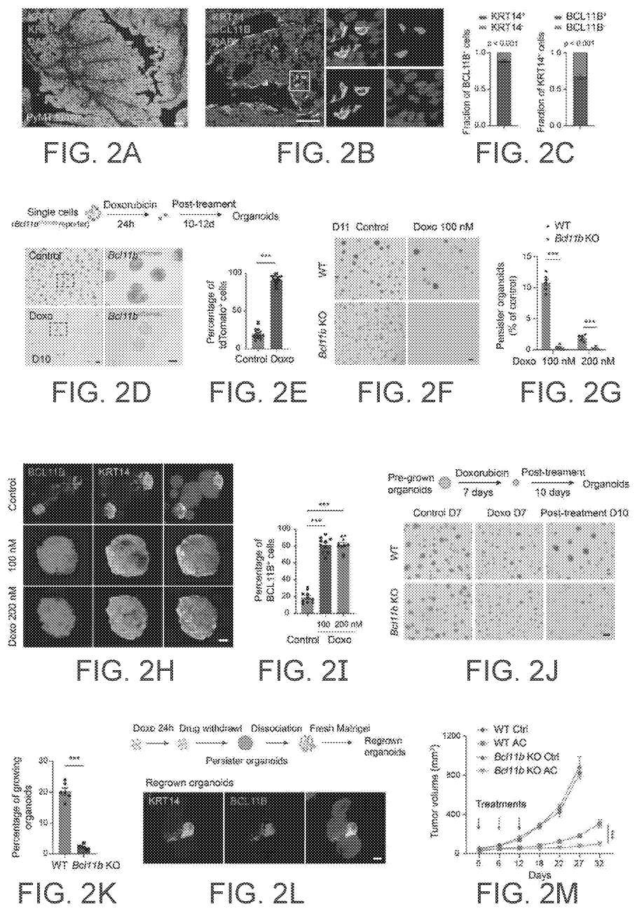

BCL11B defines a basal-like tumor subpopulation with pre-existing drug-resistance ability. HER2 has been identified as an important biomarker and treatment target for a subset of aggressive breast cancers, resulting in the development of highly successful targeted therapy. This prompted us to investigate the therapeutic significance of targeting BCL11B in addressing chemoresistance. We first experimentally examined whether the BCL11B-expressing cancer cells possess the ability to resist chemotherapeutic drugs. To this end, we utilized the widely used MMTV-PyMT (PyMT) transgenic mouse model, which has been shown to recapitulate many aspects of human breast cancer. Interestingly, despite being regarded as a luminal type of breast cancer, the primary PyMT tumor harbored a specific subset of KRT14⁺ basal-like cells, predominantly located at the tumor-stromal border and surrounding KRT8⁺ luminal tumor cells (FIG. 2A). This finding aligns with observations reported in recent studies. Of note, BCL11B was found to be expressed in a large proportion of the KRT14⁺ cells (FIG. 2, B and C). These results showed that BCL11B marks a distinctive tumor subpopulation in the PyMT tumor model, exhibiting basal-like features and resembling human BCL11B⁺ cancer cells.

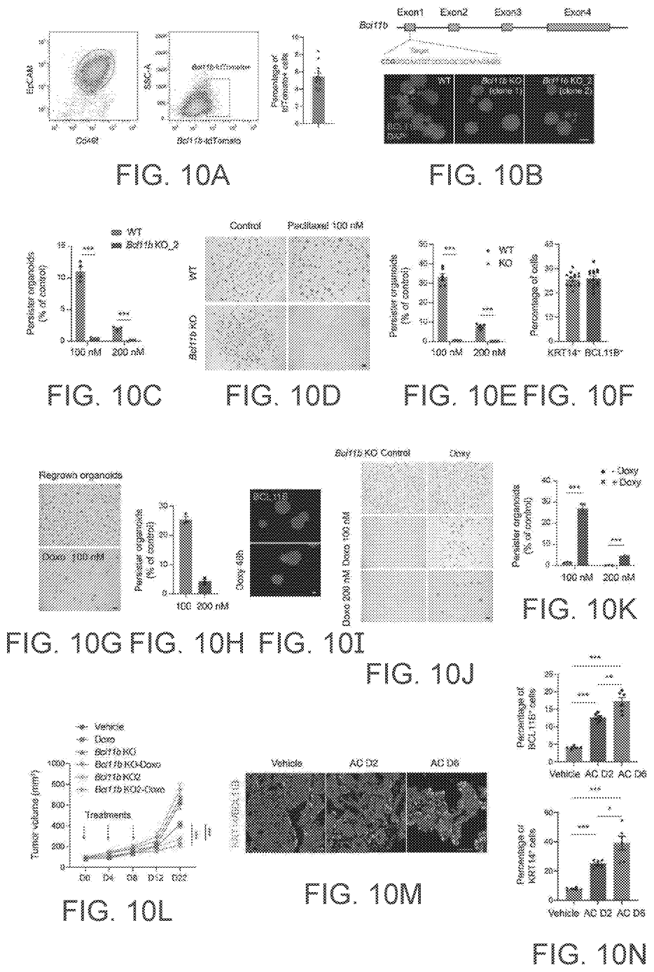

To directly test whether the BCL11B+ cancer cell subpopulation possesses a pre-existing capability to resist treatment, we generated the Bcl11b reporter model by crossing the Bcl11^{btdTomato} mice to MMTV-PyMT. Bcl11^{btdTomato}-positive cells represented a small population of tumor cells (3-8%) in the primary tumor as determined by fluorescence-activated cell sorting (FACS) analysis (FIG. 10A). We next established an ex vivo drug treatment assay starting with individual tumor cells. Briefly, single EpCAM⁺ tumor cells were sorted from PyMT-Bcl11^{btdTomato} reporter tumors and cultured in Matrigel for 24 hours in the absence or presence of doxorubicin (Doxo), one of the most widely used chemotherapy agents for treating breast cancer. The drug was then washed out, and the cells were assayed for their capacity to form tumor organoids, a model system that has been shown to retain key properties of primary tumors (39) (FIG. 2D). In the vehicle-treated control group, PyMT tumor cells robustly grew into organoids within a short period of time (3-4 days) (FIG. 2D). However, following doxorubicin treatment, only a small fraction of cells survived and gradually developed into organoids (FIG. 2D). When we analyzed the tdTomato fluorescence, we observed that the drug-resistant persister organoids were almost exclusively composed of Bcl11^{btdTomato}-positive cells, whereas only a minor subset of cells expressed tdTomato in the control organoids (FIG. 2, D and E). To explore whether Bcl11b is functionally required for the drug-resistant ability of this tumor population, we generated Bcl11b knockout (KO) tumor organoids by CRISPR/Cas9 (FIG. 10B). Bcl11b deficiency did not cause an obvious change in the survival of the tumor organoids under normal conditions but led to a near-complete elimination of persister organoids following treatment (FIG. 2, F and G, and FIG. 10C). Immunostaining analysis of the persister cells derived from WT tumor cells revealed that KRT14 and BCL11B double-positive cells were highly enriched in these organoids (FIG. 2, H and I). By contrast, the BCL11B⁺/KRT14⁺ tumor cells only constituted a subset (20%) of WT control organoids (FIG. 2, H and I). This is consistent with the finding that BCL11B marks a minor subset of cancer cells in untreated primary tumors (FIG. 10A), although its percentage in organoids moderately increases, likely due to the enrichment of less differentiated cells in vitro. Together, these results reveal that BCL11B⁺ tumor cells constitute a pre-existing drug-tolerant population that can selectively persist during chemotherapy treatment.

To study the effects of prolonged treatment, we further administered doxorubicin to pre-grown WT or Bcl11b-deficient tumor organoids for 7 days (FIG. 2J). The majority of tumor organoids in both groups demonstrated a decrease in size following the treatment, although a small number of slow-growing organoids were observed in the WT group (FIG. 2J). Notably, upon termination of the treatment, a significant portion of the organoids in the WT group resumed growth, whereas the Bcl11b-deficient organoids were lost over time (FIG. 2, J and K). We also found that the absence of Bcl11b rendered tumor cells much more sensitive to paclitaxel (taxane), another drug commonly used to treat breast cancer, highlighting the multiple-drug resistance capacity of the BCL11B-expressing cells (FIG. 10, D and E). We then asked whether the persister cells maintain a stable phenotype upon the discontinuation of drug treatment. To eliminate any potential negative impacts of residual drugs and to ensure any organoids were clonally derived from BCL11B⁺ cells, we dissociated the persister organoids into single cells and re-embedded them in fresh Matrigel. Interestingly, in the regrown organoids, the percentage of KRT14⁺/BCL11B⁺ cells dropped to a level similar to that of the pre-treatment organoids (FIG. 2L and FIG. 10F), implying the differentiation of basal-like tumor cells into luminal tumor cells. Moreover, the regrown organoids remained responsive to doxorubicin treatment due to the presence of drug-sensitive luminal cells (FIG. 10, G and H). This indicates that BCL11B⁺ cells drive a reversible cancer persistence under drug treatment.

To test whether BCL11B expression enables tumor cells to establish drug-resistant ability, we reintroduced BCL11B expression in the Bcl11b knockout organoid cells (FIG. 101). We found that the restoration of BCL11B expression induced a de novo drug-resistance phenotype in these cells (FIG. 9, J and K). To further confirm the role of BCL11B in vivo, we grew tumors from WT and Bcl11b knockout PyMT cells in mice and performed a drug treatment study. Consistent with our in vitro findings, knockout of Bcl11b dramatically increased the anti-tumor efficacy of either doxorubicin alone or the commonly used combination chemotherapy (AC, adriamycin (doxorubicin) combined with cyclophosphamide) (FIG. 2M and FIG. 10L). This result also confirms that BCL11B confers resistance to multiple chemotherapeutic drugs that are frequently used in clinics. We also observed that drug treatment induced an obvious enrichment of BCL11B⁺ cells and KRT14⁺ cells in the WT residual tumor (FIGS. 10, M and N), consistent with the role of BCL11B in mediating treatment resistance. Overall, these in vitro and in vivo observations indicate that BCL11B shapes a pre-existing tumor population that is tolerant to multiple chemotherapeutic drugs and drives a reversible cancer persistence upon treatment.

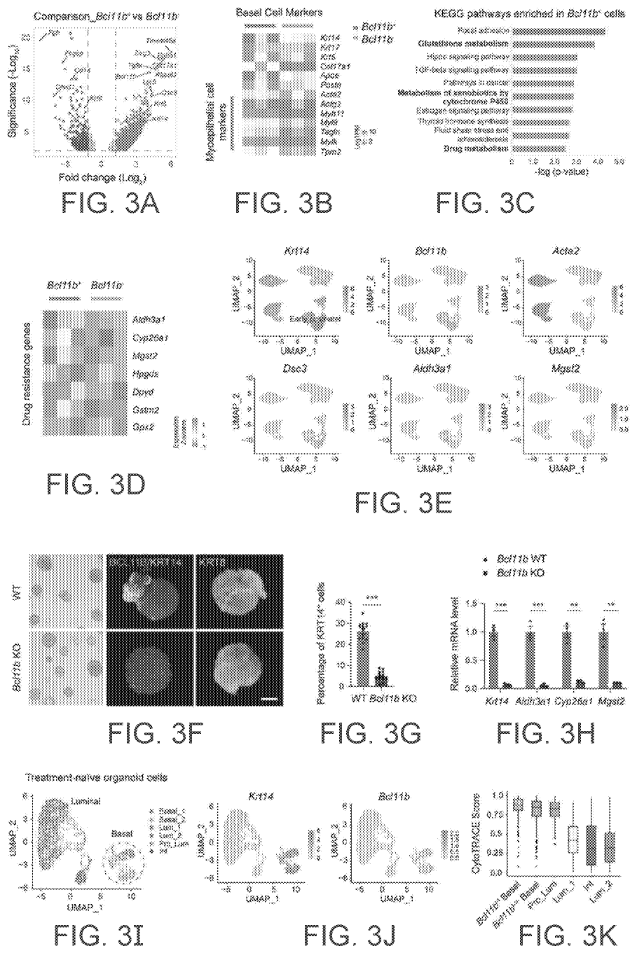

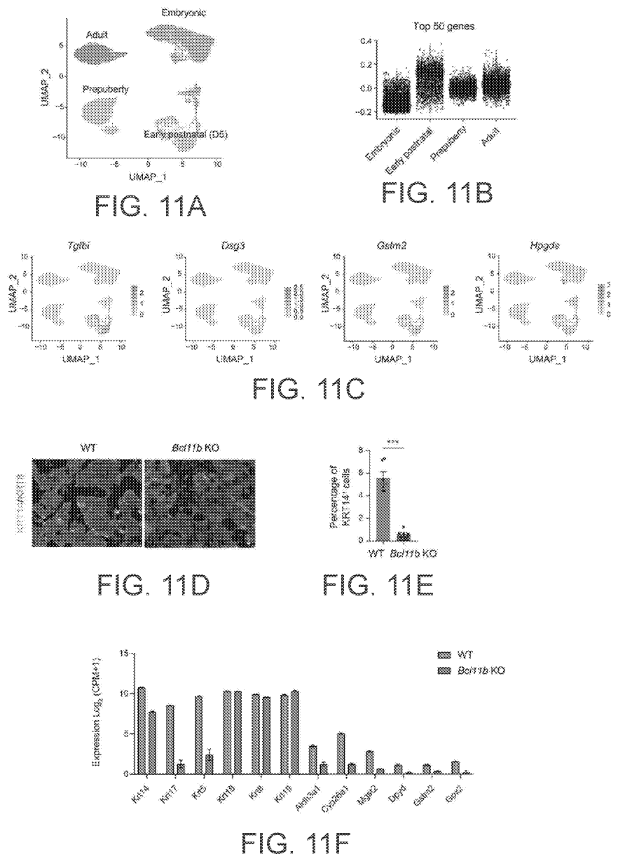

Bcl11b⁺ cell state is intrinsically associated with drug-resistant properties. To investigate how Bcl11b⁺ cells are primed to adopt a drug-resistant state, we first performed bulk RNA-seq analysis on Bcl11b⁺ and Bcl11b cells in treatment-naïve tumors. As expected, canonical basal genes such as Col17a1, Krt14, and Krt5 were highly expressed by Bcl11b⁺ cells, while luminal markers were predominantly observed in Bcl11b cells (FIG. 3A). However, many of the top enriched genes in Bcl11b+ cells, such as Dsc3, Dsg3, and Tgfbi, have not been previously linked to mammary basal cells. Conversely, markers associated with a more mature myoepithelial phenotype, including Actg2, Myh11, Myl9, and Mylk, were found to be expressed at low levels in Bcl11b+ cells (FIG. 3B), consistent with our observations in human BCL11B⁺ cells. KEGG pathway analysis further uncovered an enrichment of genes involved in glutathione (GSH) metabolism and xenobiotic metabolism in the treatment-naïve Bcl11b⁺ cells (FIG. 3C). Many of these genes such as Aldh3a1, Cyp26a1, and Mgst2 have been implicated in drug resistance (FIG. 3D). Notably, the aldehyde dehydrogenase enzyme Aldh3a1 has been demonstrated to protect the LM2 breast cancer cell line against the chemotherapeutic drugs doxorubicin, paclitaxel, and cyclophosphamide. By interrogating a recent single-cell transcriptome atlas spanning mouse mammary development stages, we also unexpectedly identified that Bcl11b and its associated gene program in cancer were specifically enriched in the rudimentary basal-like cells at the early postnatal stage (D5) (FIG. 3E and FIG. 11, A-C). The majority of basal-like cells at this early postnatal stage lacked myoepithelial markers (FIG. 3E and FIG. 11A) and represented a precursor population in an intermediate developmental stage. Interestingly, many of the drug resistance genes also showed a specific enrichment in this early basal cell state (FIG. 3E and FIG. 11C). Therefore, these results suggest that Bcl11b⁺ tumor cells mimic a precursor-like developmental state in cancer, which is inherently linked to a unique xenobiotic detoxification program that may play a physiological role in responding to external stimuli.

Given that the deficiency of Bcl11b compromised the drug resistance ability, we questioned whether Bcl11b is functionally required for maintaining the immature basal-like cell state and its associated drug resistance program. Indeed, the loss of Bcl11b resulted in a remarkable decrease in the number of KRT14⁺ tumor cells, both in vitro and in vivo, while leaving the KRT8⁺ luminal cell lineage unaffected (FIG. 3, F and G, and FIG. 11, D and E). The mRNA levels of Krt14 and the drug resistance genes, including Aldh3a1, Cyp26a1, and Mgst2, were also significantly downregulated in tumor organoids after the knockout of Bcl11b (FIG. 3H). Transcriptomic analysis of WT and Bcl11b knockout tumor organoids further confirmed that both the basal gene program and the drug resistance program were impaired upon the deletion of Bcl11b (FIG. 11F). Together, these results show that Bcl11b controls an immature basal-like cell state that is inherently linked to a unique, pre-existing drug resistance gene program and exhibits selective advantages upon chemotherapy treatment.

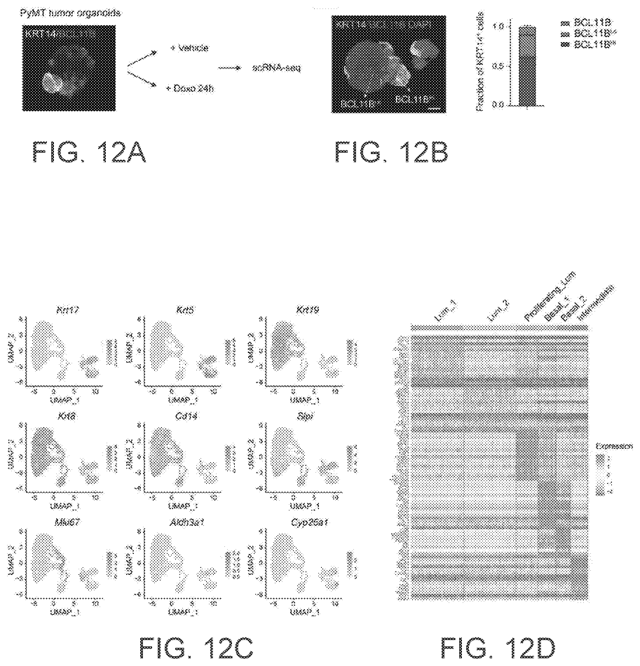

Single-cell transcriptomic characterization of the treatment-naïve tumor cells. To better understand the early transcriptional changes in individual tumor cells before and after treatment, we further conducted single-cell RNA sequencing (scRNA-seq) analysis on tumor organoids that were either untreated or treated with doxorubicin for 24 hours (FIG. 3I and FIG. 12A). After quality filtering, we generated high-quality transcriptomic data for 6,764 tumor cells (3,377 treatment-naïve and 3,387 drug-treated cells), with a median of 3,700 genes detected per cell. We first analyzed the cell populations in the treatment-naïve tumor. UMAP analysis identified two major cell lineages (Basal and Luminal) divided into six cell clusters: Basal_1, Basal_2, Luminal_1 (Lum_1), Lum_2, Proliferating_Lum, and Intermediate cells (FIG. 3I). The basal-like tumor cells exhibited a distinct transcriptional profile from the luminal cells in the single-cell dataset (FIG. 3J and FIG. 12C). The separation of basal-like cells into two closely related clusters was primarily driven by cell cycle heterogeneity, as a proportion of Basal_1 cells exhibited a moderate level of proliferation genes, while the Basal_2 cells were largely non-cycling (FIG. 3I and FIG. 12, C and D). A highly proliferating luminal cell cluster was also evident as the cells strongly expressed the proliferation markers, such as Mki67 (FIG. 3I and FIG. 12, C and D). An intermediate cell population was identified as it co-expressed multiple luminal and basal cell markers (FIG. 12, C and D).

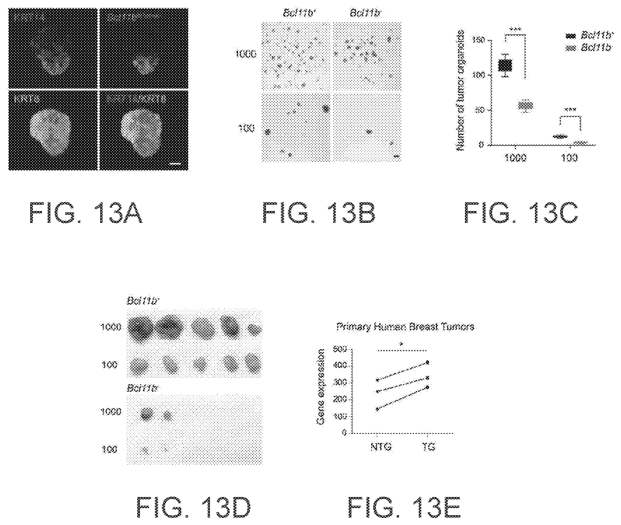

The basal-like tumor cells were marked by Krt14, and a large proportion of the cells highly expressed Bcl11b (FIG. 3J). Immunostaining analysis of tumor organoids showed that the KRT14⁺ population consists of 62% BCL11B^{High (Hi)} cells, 28% BCL11B^{Low (Lo)} cells, and 10% BCL11B-cells (FIG. 11B). Therefore, we categorized the basal-like tumor cells in the single-cell dataset into Bcl11b^{Hi cells} (mRNA level>0, 56%) and the remaining cells as Bcl11b^{Lo/-} cells. The latter group might still contain some Bcl11b⁺ cells, considering the immunostaining results and possible dropouts. Notably, the CytoTRACE analysis of the pre-treatment organoids revealed that the Bcl11b^{Hi} basal subset represented the least differentiated cells in the tumor organoids, and the basal-like tumor cells were generally less differentiated compared to luminal cells (FIG. 3K). In line with this, single Bcl11b⁺ cells can efficiently develop into tumor organoids comprising both KRT14⁺ basal-like cells and KRT8⁺ luminal cells (FIG. 13A). This suggests that, similar to the normal murine mammary gland, the Bcl11b⁺ cells retain their developmental plasticity in the tumor. Moreover, Bcl11b-expressing cells demonstrated increased tumorigenicity compared to Bcl11b⁻ cells measured by in vivo transplantation and ex vivo tumor organoid forming assays (FIG. 13, B-D). Microarray analysis of human breast tumor samples (46) confirmed that BCL11B expression was enriched in the tumorigenic-enriched population (CD44⁺CD24^{-/low}) in human breast cancer (FIG. 13E). Taken together, the transcriptomic analyses and functional assays reveal that BCL11B governs a unique immature basal-like cell state in the treatment-naïve tumor.

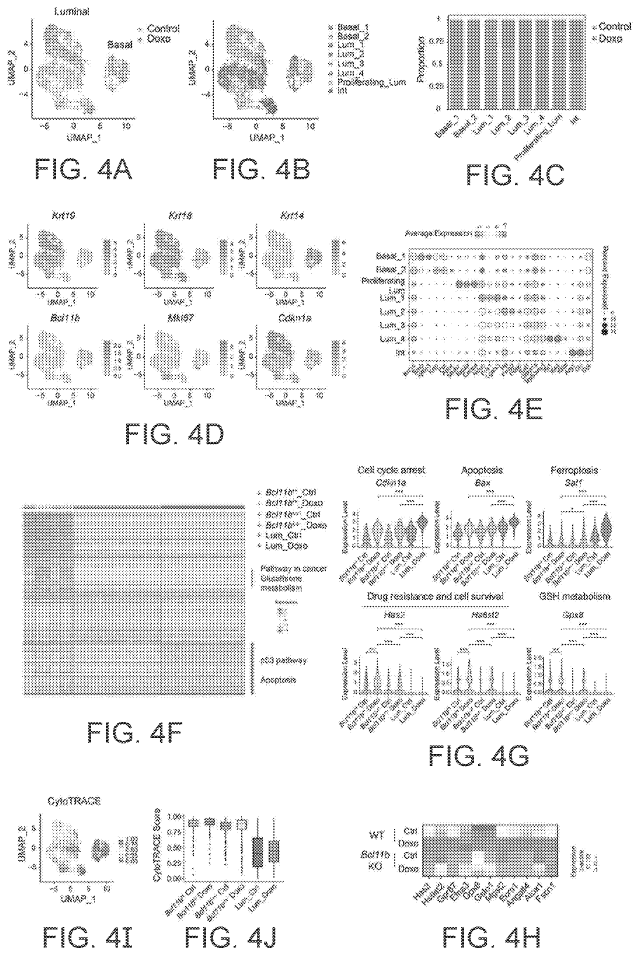

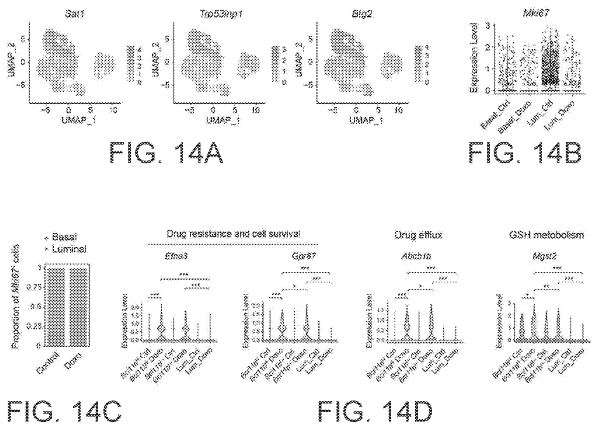

Bcl11b⁺ tumor cells demonstrate adaptive resistance capacity during treatment. We next sought to understand how different tumor subpopulations respond to drugs. To this end, we performed a combined analysis of the pre- and post-treatment datasets. Of note, the untreated and drug-treated samples were processed and sequenced together, with no batch effect observed. We discovered that while the basal-like cells from different treatment conditions were largely overlapping on the UMAP plot, the luminal cells were separated into two distinct compartments that mainly corresponded to their treatment statuses (FIG. 4, A and B). Consistently, the untreated and drug-treated basal-like cells were almost evenly spread across the basal cell clusters, whereas drug-treated luminal cells were largely confined to two specific luminal clusters: Lum_3 and Lum_4 (FIG. 4C). Analysis of the cluster marker genes revealed that Lum_3 and Lum_4 cells displayed significant enrichment of p53, apoptosis, and interferon response-related pathways, while none of these pathways were enriched in basal-like tumor cells (FIG. 4, D and E, and FIG. 14A). Moreover, the post-treatment luminal compartment showed a dramatic loss of Ki67⁺ proliferating cells (FIG. 4, C and D, and FIG. 14B). By contrast, a considerable portion of basal cells retained Ki67 expression after the treatment (FIG. 14, B and C). These results suggest that basal-like cells were only moderately impacted by the drug treatment.

To gain a deeper insight into the transcriptional responses that occurred in different tumor subpopulations, we re-grouped the cells into six categories: Bcl11b^{Hi} basal_Ctrl, Bcl11b^{Hi} basal_Doxo, Bcl11b^{Lo/-} basal_Ctrl, Bcl11b^{Lo/-} basal_Doxo, Lum_Ctrl, and Lum_Doxo. As anticipated, pre- and post-treatment basal-like tumor cells were transcriptionally similar, while the drug treatment induced a drastic gene expression change in luminal cells featured by the activation of p53 and apoptosis pathways (FIG. 4F). Indeed, genes associated with cell cycle arrest, apoptosis and ferroptosis, such as Cdkn1a, Btg2, Bax and Sat1 (47-50), were strongly induced in the luminal compartment following the treatment (FIG. 4G). We also noticed that a unique group of genes pre-existing in the basal compartment showed a specific upregulation in these cells upon treatment (FIG. 4F). The GSH metabolism pathway and pro-survival cancer pathways are among the top enriched KEGG pathways. The above-discussed GSH metabolism pathway contributes to the detoxification of drugs and reactive oxygen species and has been found to be specifically enriched in chemoresistant squamous cell carcinoma stem cells and radiation-resistant breast cancer stem cells. Moreover, many cancer related genes within this gene group have previously been linked to various drug resistance and cell survival mechanisms, such as the activation of oncogenic signaling cascades (Has2, Gpr87, Ecm1, Angptl4), heparan sulfate transferase-mediated drug resistance (Hs6st2), drug efflux (Abcb1b), and enhanced anti-apoptosis functions (Efna3 (60)) (FIG. 4G and FIG. 14D).

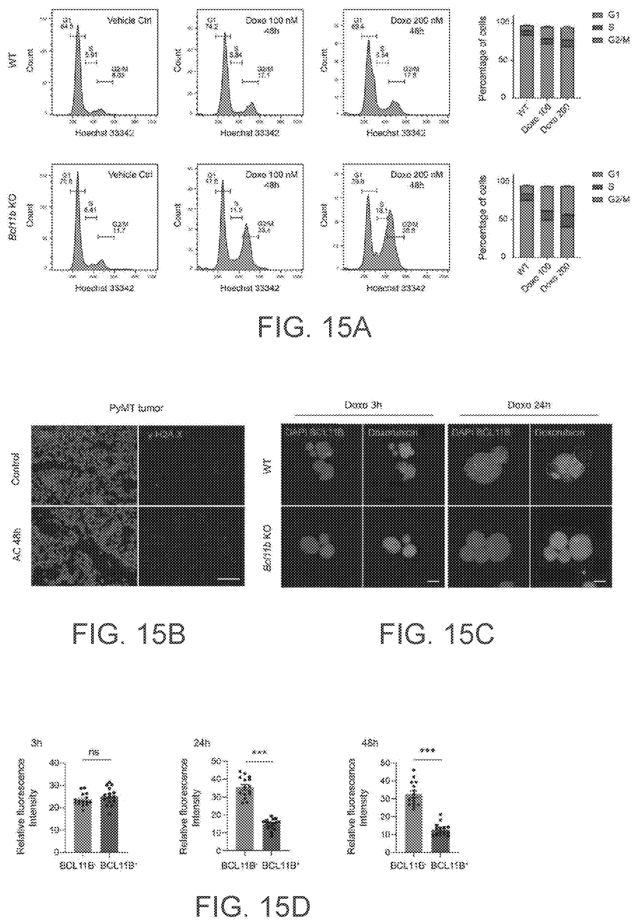

While the Bcl11b^{Hi} and Bcl11b^{Lo/-} basal cells demonstrated certain transcriptional similarities, likely due to both being basal cells at different maturity stages, the Bcl11b^{Hi} subset displayed a clearly stronger upregulation of the drug-resistance program compared to the Bcl11b^{Lo/-} tumor cells (FIG. 4F). Consistently, representative drug-resistant genes and GSH metabolism genes exhibited higher upregulation in the Bcl11b^{Hi} subset in response to treatment (FIG. 4G and FIG. 14D). Of note, the expression of these drug resistance gene programs was dependent on Bcl11b, as drug treatment was unable to induce the majority of these genes in the Bcl11b-deficient tumor cells (FIG. 4H). This underscores the essential functional role of Bcl11b in maintaining the basal cell lineage and enabling the drug-resistance responses. Intriguingly, the independent CytoTRACE analysis revealed drug-treated Bcl11b^{Hi} cells as the least differentiated cells among the tumor subpopulations (FIG. 4, I and J). This indicates that the Bcl11b^{Hi} subset contains cells in an even more undifferentiated state in response to treatment, thus increasing cell fitness under pressure. Consistent with these analyses, a marked cell cycle arrest was noted in tumor organoids treated with doxorubicin for 24 hours, and the knockout of Bcl11b significantly magnified the cell cycle arrest effects (FIG. 15A). Interestingly, the drug induced minimal DNA damage in the tumor cells compared to the notable cell cycle arrest induced (FIG. 15B). Moreover, in line with the involvement of multiple drug detoxification programs, there is an obvious clearance of intracellular doxorubicin in BCL11B⁺ cells as opposed to BCL11B⁻ cells within 24 hours of administration (FIG. 15, C and D). Collectively, these results suggest that Bcl11b not only controls a pre-existing drug-resistance machinery but also enables an adaptive gene network involving numerous drug-resistant and prosurvival pathways upon treatment, offering comprehensive defense against chemotherapy.

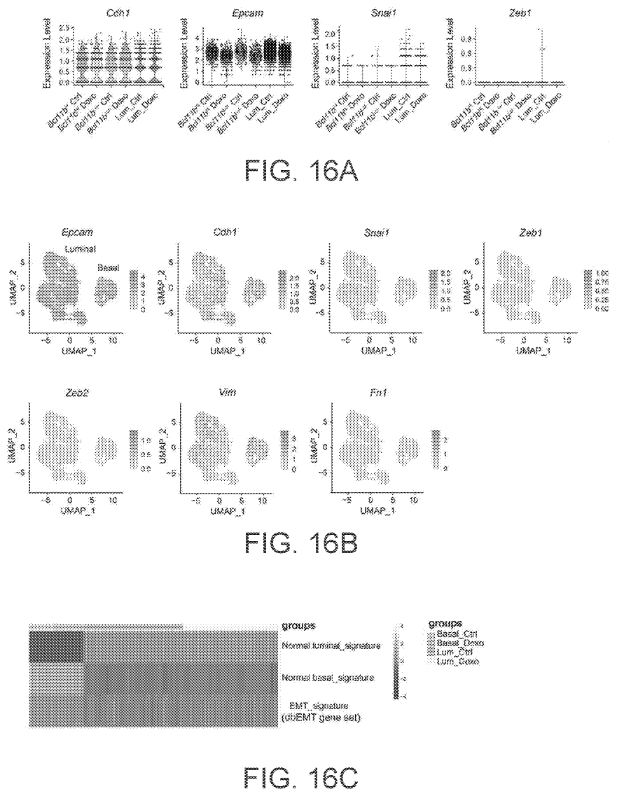

The Epithelial-to-Mesenchymal Transition (EMT) has been frequently linked to treatment resistance in cancer. Studies have demonstrated that breast cancer cells in a more mesenchymal state display increased resistance to chemotherapy. The EMT process typically involves the loss of epithelial markers (Epcam and Cdh1) and the activation of EMT-specific master regulators, particularly Snai1 and Zeb1. Here in the tumor organoids, we found that the basal-like tumor cells highly expressed epithelial cell marker genes (Epcam and Cdh1), and were largely negative for the EMT activators, Snai1 and Zeb1 (FIG. 16, A and B). Other EMT markers associated with a more mesenchymal phenotype, such as Vim and Fn1, were either lowly expressed in the tumor cells or displayed a non-specific expression pattern among tumor cells (FIG. 16B). FACS analysis has also confirmed that the basal-like tumor cells stained positive for EpCAM (FIG. 9A). Moreover, gene set enrichment analysis revealed that the normal basal cell signature was significantly enriched in the basal-like tumor cells, while the EMT gene set was expressed at comparable levels between basal and luminal cells (FIG. 16C). This implies that the basal-like tumor cells exhibit more similarities to normal basal cells as opposed to cells that have undergone EMT activation. It should be noted that a small number of luminal tumor cells displayed expressions of Snai1, Zeb1, and Zeb2 (FIG. 16, A and B), which is consistent with the idea that the EMT state might arise from luminal cells at a late stage. Nevertheless, our results clearly show that the Bcl11b⁺ drug-resistant tumor cells maintain epithelial cell identity and are distinct from the previously reported chemoresistant EMT states.

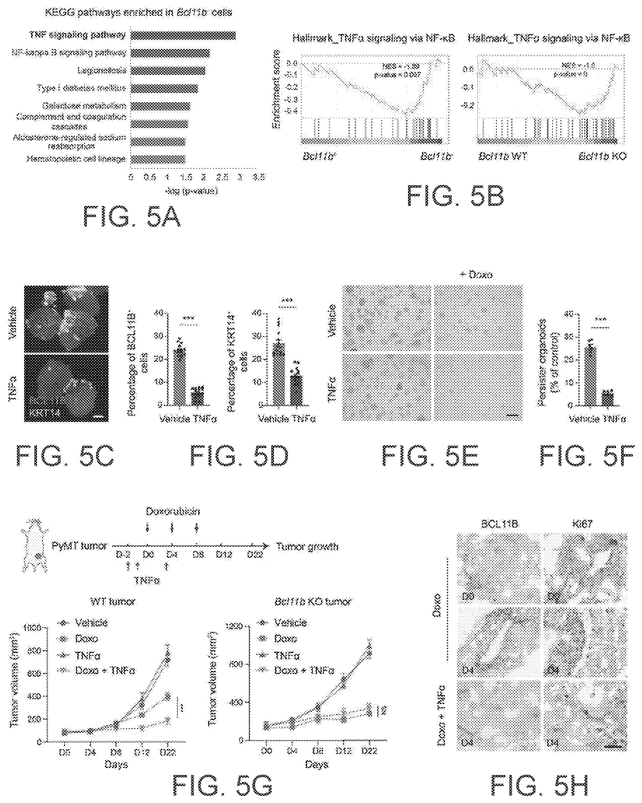

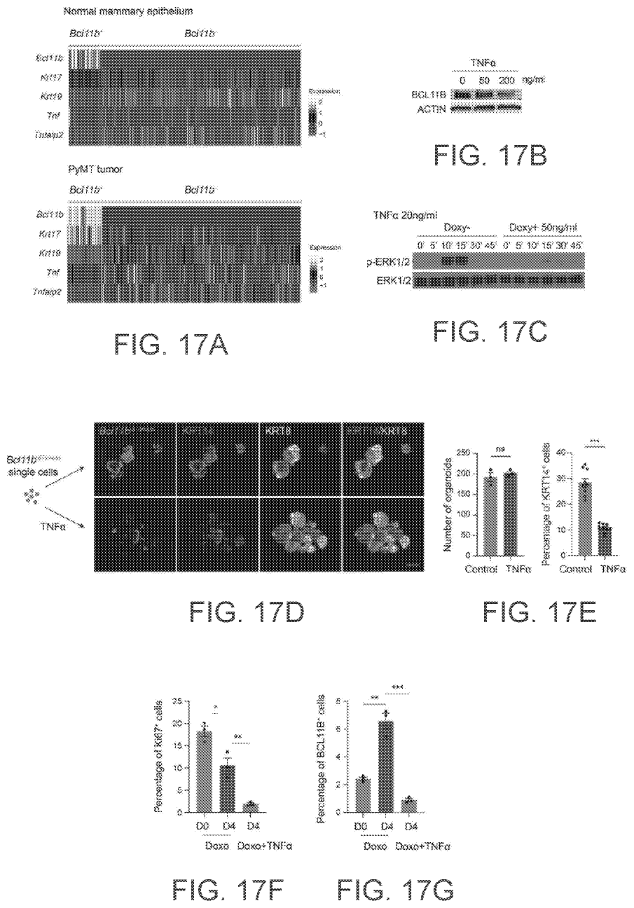

The emergence of persister cells is repressed by TNFα treatment. Because the BCL11B⁺ cancer cells express various disparate drug-resistant programs that would require multiple therapeutics for targeting, we therefore asked whether there was a way to directly inhibit the BCL11B⁺ cell state and whether this could be a therapeutic approach against chemoresistance. To search for functional regulators of the Bcl11b⁺ cells, we analyzed the bulk RNA-seq data of Bcl11b⁺ and Bcl11b⁻ cells and identified TNFα signaling as the top-enriched pathway in Bcl11b⁻ cells (FIG. 5A). This finding was corroborated by the analyses of scRNA-seq data of tumor organoids and bulk RNA-seq data of WT and Bcl11b knockout tumor cells (FIG. 5B). TNFα, a pro-inflammatory factor, plays a physiological role in regulating mammary gland development and can signal through diverse pathways including nuclear factor NF-κB and MEK/ERK pathways. Notably, single-cell analyses of normal mammary epithelium and the tumor organoid revealed an opposite expression pattern of Tnf and Bcl11b (FIG. 17A). Tnfaip2, which is a primary response gene of TNFα, is also specifically enriched in Bcl11b negative cells (FIG. 17A). Thus, we hypothesize that TNFα, which can be secreted by luminal cells and niche cells, could function as a natural inhibitor of the Bcl11b⁺ cell state.

TNFα treatment caused a marked decrease in BCL11B expression in the murine mammary epithelial cell line, Comma-Dβ cells (FIG. 17B). Interestingly, induced overexpression of BCL11B, in turn, dramatically attenuated TNFα-induced ERK activation in these cells (FIG. 17C). This revealed an unexpected reciprocal inhibition between TNFα signaling and BCL11B in mammary epithelial cells. We then exposed PyMT tumor organoids to TNFα treatment and observed a significant reduction in the number of BCL11B⁺ tumor cells within a few days (FIG. 5, C and D). The percentage of KRT14⁺ cells also decreased accordingly in the post-treatment tumor organoids (FIG. 5, C and D). We next treated single Bcl11^{btdTomato}-positive tumor cells with TNFα and tracked the fates of these single cells. In the absence of TNFα, single BCL11B⁺ cells developed into tumor organoids with a prominent population of KRT14⁺ basal-like cells among the luminal cells (FIG. 17D). Treatment with TNFα did not compromise the organoid formation efficiency, but notably decreased the proportion of KRT14⁺/BCL11B⁺ basal-like cells within the formed organoids (FIG. 17, D and E). This indicates a TNFα-induced basal to luminal lineage shift in the tumor cells. Importantly, pre-treatment with TNFα effectively decreased the emergence of persister cells (FIG. 5, E and F), showing that impairing the BCL11B⁺ cell state can avert the development of drug resistance in breast cancer.

To assess the therapeutic potential of the TNFα combination treatment in vivo, we administered three doses of TNFα in conjunction with three cycles of doxorubicin to tumor-bearing recipient mice (FIG. 5G). TNFα injection was given at a tolerated dose and was started prior to the chemotherapy treatment. TNFα administration alone did not induce noticeable alterations in tumor growth or the overall health of the mice. However, when combined with doxorubicin, TNFα remarkably enhanced the efficacy of chemotherapy in suppressing the growth of the WT tumors (FIG. 5G). The combination of TNFα and doxorubicin had no synergistic effects on Bcl11b-deficient tumor cells, suggesting that TNFα mainly functioned through targeting the BCL11B⁺ cells (FIG. 5G). Analysis of the on-treatment and post-treatment samples revealed that doxorubicin exposure caused a significant decrease in cell proliferation in the tumor, although residual zones of Ki67⁺ cells were consistently observed during the treatment (FIG. 5H and FIG. 17F). BCL11B⁺ cells were also found to be enriched in these residual lesions (FIG. 5H and FIG. 17G). In contrast, treatment with a combination of TNFα and doxorubicin eliminated both BCL11B⁺ cells and residual proliferating cells (FIG. 5H and FIG. 17, F and G). Together, these results reveal that TNFα treatment in combination with chemotherapy can strongly enhance chemotherapy efficacy by impairing the BCL11B⁺ cell state and preventing the emergence of cancer persisters.

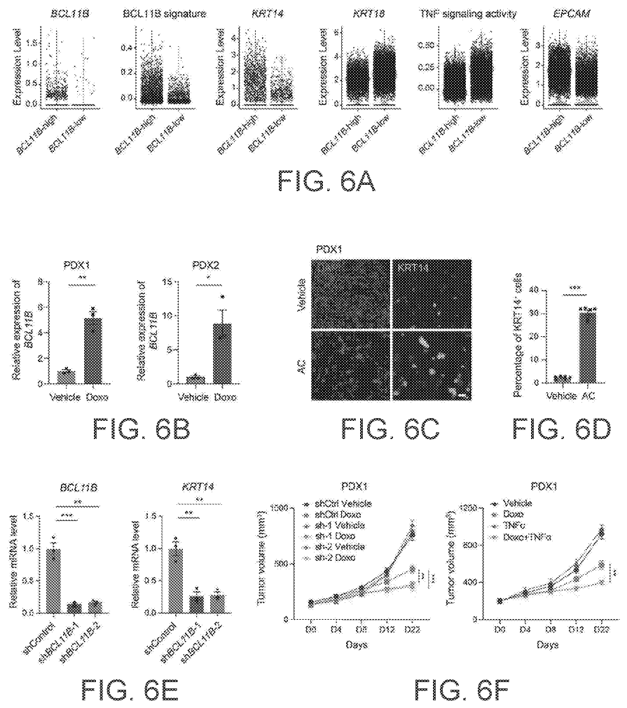

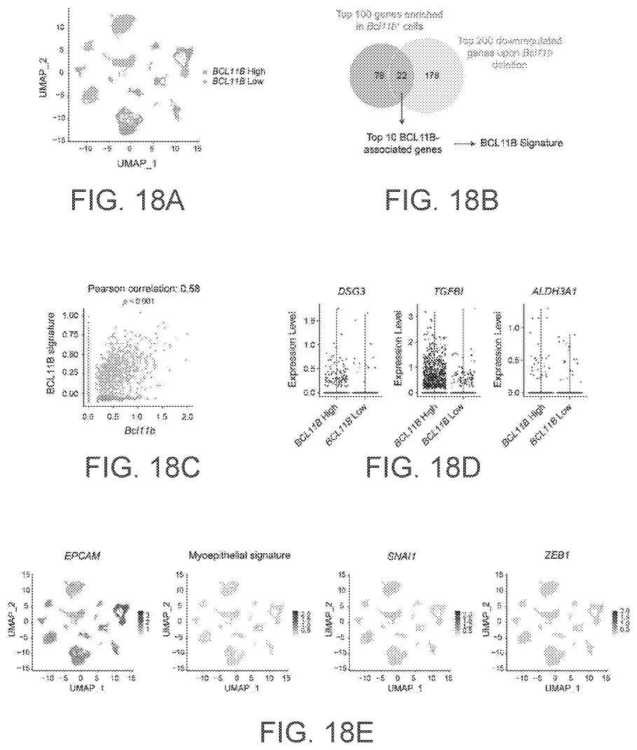

BCL11B promotes treatment resistance in human breast cancer. To assess the implications of our mouse tumor findings for human breast cancer, we first revisited the single-cell breast cancer atlas. Considering the potential dropout of BCL11B expression in single-cell datasets due to its low expression levels in cells, we broadly divided tumor cells into BCL11B-high and BCL11B-low compartments based on the average expression level of BCL11B in each Seurat cell cluster (FIG. 18A). As expected, BCL11B expression was enriched in the BCL11B-high compartment (FIG. 6A). We next defined a ‘BCL11B signature’ that consists of the top 10 BCL11B-associated genes (Ephb1, Dsg3, Col17a1, Tgfbi, Cntfr, Ngfr, Cyp26a1, Postn, Pnpla1, and Id4) from the mouse tumor data. Genes that were highly expressed in Bcl11b⁺ cells and were markedly downregulated by Bcl11b deletion were identified as BCL11B-associated genes (FIG. 18B). We then validated the BCL11B signature in our scRNA-seq dataset of the tumor organoids and found a strong correlation between Bcl11b expression and the BCL11B signature (FIG. 18C). Upon examining the cancer epithelia in the breast cancer atlas, we observed that the BCL11B signature and KRT14 were significantly enriched in the BCL11B-high compartment, while KRT18 was expressed at a lower level in the BCL11B-high cells (FIG. 6A). The genes associated with the rudimentary basal-like cell state and the drug resistance programs, including Dsg3, Tgfbi, and Aldh3a1, were also enriched in the BCL11B-high tumor population (FIG. 18D). Moreover, the BCL11B-high tumor compartment showed no enrichment in myoepithelial markers and displayed a lower level of TNFα signaling activity (FIG. 6A and FIG. 18E). Together, these results suggest that the TNFα/BCL11B⁺ immature cell state/drug-resistance axis is largely conserved in human breast cancer. We also observed that the majority of cancer cells were positive for EPCAM (FIG. 6A and FIG. 18E) and demonstrated low levels of SNAI1 and ZEB1, two critical EMT factors in breast cancer (FIG. 18E), consistent with the findings in mouse tumors.

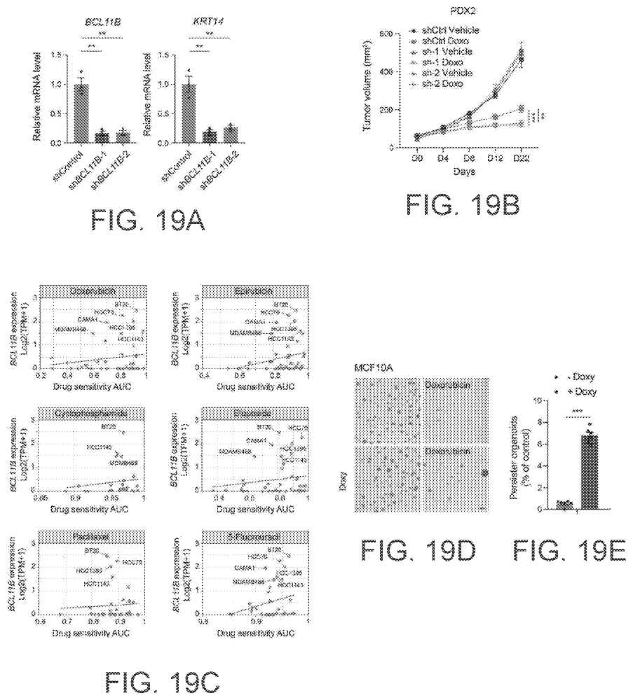

To directly assess the functional role of BCL11B in promoting drug resistance in human breast cancer, we further utilized the breast cancer patient-derived xenograft (PDX) models that were previously established in our laboratory. High levels of BCL11B⁺ tumor cells were detected in 2 out of the 5 triple-negative xenograft models we tested, consistent with our findings that BCL11B is expressed in a subset of human breast cancers. Chemotherapeutic treatment induced upregulation of BCL11B mRNA levels in both PDX tumor lines, which was corroborated by the observation that KRT14⁺ cells were significantly enriched in the post-treatment tumors (FIG. 6, B-D). We then knocked down BCL11B in the PDX models and found a corresponding decrease in the expression of KRT14 (FIG. 6E and FIG. 19A). Similar to the findings in mice studies, the anti-tumor efficacy of doxorubicin was significantly augmented upon the inhibition of BCL11B through shRNA-mediated knockdown in the PDX models (FIG. 6F and FIG. 19B). We also confirmed that TNFα treatment notably enhanced the anti-tumor effects of chemotherapy on WT PDX tumor cells (FIG. 6F). We next extended our analysis to human breast cell lines. Analysis of the breast cancer cell lines in the DepMap database showed that BCL11B displayed significant variability in expression levels, with high expression in a subgroup of the cell lines (FIG. 19C), including BT20, HCC70, and HCC1395. Interestingly, almost all of these BCL11B-high cancer cell lines demonstrated a notable resistance to commonly used chemotherapeutic drugs, as revealed in the GDSC database (FIG. 19C). To verify whether BCL11B can initiate the drug-resistance programs in the human context, we induced an overexpression of BCL11B in MCF10A normal human breast cells. These cells have minimal gene mutations and serve as a model for both normal and malignant mammary epithelial cell biology. The ex vivo drug treatment assay showed that the ectopic expression of BCL11B induced de novo emergence of persister organoids in response to doxorubicin treatment (FIG. 19, D and E). Overall, our studies on patient-derived tumor models revealed that BCL11B⁺ cancer cells functionally promote resistance against chemotherapy in human breast cancer.

Different mechanisms mediating chemotherapy resistance in cancer have been proposed, but a key unresolved question is why tumors display great variability in drug responses. A well-appreciated theory of drug-tolerant persister proposes that a subpopulation of tumor cells can enter a slow-cycling, drug-tolerant state under drug pressure to evade treatment. However, it remains ambiguous whether cancer persisters result from the selection of pre-existing tumor states or emerge non-selectively from tumor cells, making it challenging to explain the reasons behind the significant variation in tumor responses to chemotherapy. Evidence from recent lineage-tracing studies in colorectal cancer and skin cancer reveals that certain pre-existing stem-like tumor subpopulations persist during chemotherapy and drive tumor regeneration, supporting the notion that the emergence of cancer persisters can be driven by pre-existing mechanisms.

Here in breast cancer, we revealed that the pre-existing BCL11B⁺ cancer cells can selectively transition to a persister state following treatment, benefiting from their fitness advantages conferred by their innate drug resistance machinery. Interestingly, the BCL11B⁺ cancer cells also exhibit a notable capacity to adapt transcriptionally in response to drug treatment, whereas the luminal tumor cells are prone to entering cell cycle arrest and apoptosis under drug stress. Therefore, our results support a model in which pre-existing tumor populations with inherent fitness advantages can preferentially and actively evolve a resistant state through transcriptional adaptation mechanisms. This is distinct from the general notion that drug resistance emerges through the passive selection of pre-existing subpopulations or through transcriptional adaptations that occur stochastically among tumor cells. Notably, we have also shown that tumors with populations of BCL11B⁺ cells first decrease in size after therapy, but the minority population of BCL11B⁺ cells will regenerate a tumor with a mixed population of cells, which will once again shrink after the same therapy (FIG. 2L, and FIG. 9, G and H, and FIG. 12A). This provides an alternative model explaining why some patients initially respond to therapy but their tumors continue to regrow after a drug holiday and exhibit shrinkage again upon the reintroduction of the same therapy.

As a zinc finger transcriptional factor and a component of various chromatin-remodeling complexes such as NURD and SWI/SNF, BCL11B can regulate different gene programs depending on the cellular context and cell type. BCL11B is essential for fetal development and progenitor maintenance in multiple tissues, including the mammary gland, epidermis, and neural tissues. The absence of BCL11B in these tissues may result in developmental deficiencies, such as neurological disorders, while not being linked to cancer initiation. In contrast, BCL11B directs T cell lineage commitment, while the depletion of BCL11B impairs T cell maturation and can lead to the development of T-cell malignancies. On the other hand, overexpression of wild-type BCL11B is observed in multiple cancers, including T cell leukemia. The recent discovery of the role of BCL11B in DNA repair in T cells offers a possible explanation for its paradoxical roles in T cell malignancies. Inactivating genetic alteration in BCL11B causes mutation accumulation due to disrupted DNA repair but overexpression of the normal BCL11B protein protects cancer cells against oxidative DNA damage. Loss-of-function mutations in BCL11B are rare in human breast cancers (less than 1%) compared to T-cell acute lymphoblastic leukemia (18%), indicating its context-dependent role in different cancers. Indeed, here in breast cancer, we did not observe that BCL11B utilizes the DNA repair machinery to mediate treatment resistance, as the chemotherapy drugs we studied induce minimal DNA damage in these cells. Instead, the chemotherapy drugs mainly induce cell cycle arrest and subsequent cell death in the cells. BCL11B controls a complex drug resistance gene program that facilitates drug elimination and enhances cell survival, thereby exposing a new role of BCL11B in safeguarding cancer cells.

Breast cancer is a disease that is widely recognized for its significant level of heterogeneity. The identification of biomarkers such as HER2/NEU, which can pinpoint a specific subset of patients who can benefit from targeted therapy, can be critical for achieving successful treatment outcomes. A number of other marker genes that are enriched in less differentiated breast cancer cells have been identified, but many of them lack specificity, limiting their predictive value and potential for therapeutic targeting. Here, by systematically interrogating single-cell atlases of human breast cancer using the CytoTRACE algorithm, we identified a previously unrecognized BCL11B⁺ immature cancer cell state that is naturally impervious to a multitude of chemotherapeutic drugs, resulting in a higher possibility of chemotherapy resistance. Therefore, our findings identify BCL11B as a new biomarker for subtyping breast cancer and predicting chemotherapy resistance in some patients. Utilizing BCL11B as an immunohistochemical prognostic marker for assessing biopsy samples prior to the start of chemotherapy shows great promise and feasibility.

Notably, directly targeting this master regulator of the drug-resistant cell state through a therapeutic agent, TNFα, greatly enhances chemotherapy efficacy in the subset of breast cancer tumors with a population of BCL11B⁺ cancer cells. In line with this, early research has illustrated the success of utilizing TNFα in conjunction with chemotherapy for the treatment of advanced soft tissue sarcomas. Treatment of breast cancer patients, particularly TNBC patients with cancers localized to the breast and regional lymph nodes with preoperative chemotherapy including Taxanes and Anthracyclines results in increased survival, especially in patients whose breast tumors demonstrate a major response to therapy. Our results show that in tumors with populations of BCL11B⁺ cells, adding TNFα prior to the start of Taxanes or Anthracyclines treatment can dramatically improve treatment response, both in vitro and in vivo. Thus, our results suggest that BCL11B is useful as a biomarker for identifying a subset of human breast cancers with a higher risk of developing chemoresistance that benefit from precision therapy combining chemotherapy with TNFα.

### Methods

Mice. MMTV-PyMT (stock #002374 and 022974), NOD scid gamma (NSG, stock #005557), and Krt14-cre (stock #018964) mice were purchased from Jackson Laboratories. Bcl11b^{flox/flox} mice and Bcl11^{btdTomato} knock-in reporter mice were generously provided by Mark Leid's lab and Pentao Liu's lab, respectively. For xenograft studies, 8-12 weeks old immunocompromised NSG females were used. All animal procedures were conducted in accordance with a protocol approved by the Stanford University APLAC committee. Mice were maintained in-house under aseptic sterile conditions. Mice were administered with autoclaved food and water.

Cell lines. MCF10A and 293T cells were obtained from ATCC. MCF10A cells were grown in DMEM (Invitrogen) supplemented with 5% Horse Serum (Invitrogen), 20 ng/ml EGF (Invitrogen), 0.5 mg/ml Hydrocortisone (Sigma), 100 ng/ml Cholera Toxin (Sigma), 10 μg/ml Insulin (Sigma), and 1% PSA (Invitrogen). 293T cells were grown in DMEM (Invitrogen) supplemented with 10% FBS (Hyclone), Glutamax (ThermoFisher Scientific), sodium pyruvate (Life Technologies), and 1% PSA (Invitrogen). Comma D beta cell line was cultured in DMEM-F12 (Invitrogen) supplemented with 2% Fetal Bovine Serum (Hyclone), 1% PSA (Invitrogen), 10 ng/ml EGF (Invitrogen) and 5 μg/ml Insulin (Sigma). All cells were incubated at 5% CO₂ and 37° C. None of the cell lines used are listed in the database of commonly misidentified cell lines maintained by ICLAC. Cell lines have not been authenticated but all cell lines used were passaged less than 10 times from when the original cells from the vendors were thawed.

Preparation of single-cell suspension and flow cytometry. For human samples, informed consent was obtained, and human normal breast and breast tumor specimens were collected according to guidelines from Stanford University's Institutional Review Board (4344). Mouse primary mammary tumors were obtained from 12-16 weeks-old virgin female MMTV-PyMT mice. Mouse or human xenograft samples were obtained from NSG recipient mice that had been transplanted with tumor cells. Tumor samples were dissected and processed according to the published protocol with minor modifications. In brief, samples were mechanically dissociated into small pieces (1-2 mm³) with a razor blade and then digested at 37° C. with 1,500 U collagenase and 500 U hyaluronidase (Stem Cell Technology) in Advanced DMEM/F-12 (Thermo Fisher Scientific) supplemented with 2% FBS (Hyclone), and 1% PSA (Invitrogen) for 3 hours with hourly pipetting. After digestion, the reaction was neutralized by adding FACS staining buffer (HBSS+2% FBS+1% PSA) and spun down at 1500 rpm for 5 min. The pellet was then treated with 5 mL of ACK lysis buffer (Lonza) on ice for 5 min to deplete red blood cells. The sample was spun down and the cells were further digested by 4 ml 0.25% Trypsin-EDTA (GIBCO) supplemented with 1,000 U DNase for 10 min at 37° C. Dissociated cells were filtered by a 40 μm strainer and washed with FACS staining buffer. For FACS analysis and sorting, single cells were stained with CD45 (Biolegend), CD31 (Biolegend), Ter119 (Biolegend), CD49f (Biolegend),

EpCAM (Biolegend), and CD24 (Biolegend) with appropriate conjugated fluorophores for 20 min on ice. The cells were then washed and resuspended in FACS staining buffer containing DAPI (1 μg/ml). Stained cells were analyzed and sorted on a FACSAria II (BD Bioscience) instrument with a 100 μm nozzle.

Organoid culture. 20 ul of growth factor reduced Matrigel (BD Bioscience) was added to a well of a round-bottom 96-well plate and allowed to solidify at 37° C. for 10 min. FACS-sorted tumor cells were then resuspended in 150 μl organoid culture media (Advanced DMEM/F12+2% FBS+PSA+N2+B27+10 mM HEPES+EGF (10 ng/ml, Invitrogen)+Rspo1 (250 ng/ml, R&D)+ROCK inhibitor Y27632 (10 μM, Sigma)+Noggin (100 ng/ml, R&D)), and overlaid on top of the Matrigel. Organoids were grown in humidified tissue culture incubators at 37° C. in 5% CO₂ and were supplemented every two or three days with fresh media. For passaging, organoids were first incubated in pre-chilled cell recovery solution (Corning) on ice for 20 min and then dissociated into single cells by trypsin digestion.

CRISPR-mediated genome editing in tumor organoids. Guide RNA targeting the exon 1 of Bcl11b (CCGGGCAATGTCCCGCCGCAAACAGG) was cloned into Cas9 containing lentiCRISPR v2 vector. Lentivirus production and transfection were performed as described above. After infection, single tumor cells were plated on Matrigel for organoid culture, followed by selection with puromycin. Single organoid clones were then isolated and expanded. Genomic DNA was isolated from the selected clone and the targeted mutation in the Bcl11b locus was confirmed by Sanger sequencing.

Transplantation. Dissociated single cells from tumors were resuspended in 100 μl of media (organoid culture media+50% of Matrigel) and injected into the nipple of female NSG mice at the fourth mammary fat pad. For lentivirus-mediated BCL11B knockdown transplant assay, dissociated xenograft cells were transduced overnight in organoid culture media with 20 MOI. of lentivirus. The next day, PDX cells were transferred to Matrigel and cultured in organoid media+Neuregulin 1 (5 nM, PeproTech)+A83-01 (500 nM, Tocris)+SB202190 (500 nM, Sigma). 72 hours after transduction, organoids were selected with 1 μg/mL puromycin. Transduced organoid cells were grown in the selection media for 4-5 days and then transplanted to NSG recipient mice as described above.

In vivo treatment. Tumor-bearing NSG mice were randomized into control and treatment groups when the tumor width reached 3-5 mm for drug treatment. Saline, doxorubicin (1 mg/kg body weight, every 4 days), or doxorubicin (1 mg/kg) combined with cyclophosphamide (60 mg/kg) (every 6 days) were given to the mice by intraperitoneal injection for the duration indicated in the regimen. For experiments involving TNFα treatment, murine or human recombinant TNFα (100 μg/kg, PeproTech) was administered by intraperitoneal injection as indicated in the regimen. Health and body weight were monitored every 2-3 days over the course of treatment, and tumor growth was measured every 3-4 days until the endpoint of the experiment.

RNA isolation and real-time PCR. Total RNA was extracted with RNeasy Plus Micro Kit (Qiagen) and reverse transcribed to cDNA using SuperScript III First Strand Synthesis kit (Life Technologies) according to the manufacturer's instructions. Real-time PCR was performed on the 7900HT Fast Real-Time PCR System (Applied Biosystems) or the QuantStudio 7 PRO Real-Time PCR System (Thermo Fisher) using Power SYBR Green PCR Master Mix (Life Science Technologies). Gapdh or Actb were used to normalize the expression values. RNA-sequencing and data processing 20-100 ng total RNA was used to generate barcoded RNA-seq libraries using the NEBNext Single Cell/Low Input RNA Library Prep Kit for Illumina (New England Biolabs) following the manufacturer's instructions. Libraries were sequenced on a NextSeq 550 (Illumina) or HiSeq 2500 (Illumina) to generate 75 bp SE reads or 76 bp PE reads. The quality of trimmed Fastq reads was assessed using FastQC. HISAT2 was then used to align the RNA-seq reads to the mouse reference genome (mm10), followed by gene count quantification using htseq-count. Alternatively, RNA-seq results were aligned to the mouse reference genome (mm10) and quantified using Salmon. Count matrices were analyzed with DESeq2 or Degust. Heatmaps were generated using Morpheus software (Broad Institute). KEGG pathway analysis was performed using EnrichR.

Single-cell RNA sequencing. Pre-grown tumor organoids were treated with or without doxorubicin (100 nM) for 24 hours. After the treatment, tumor organoids were recovered from Matrigel and digested into single cells by 0.25% Trypsin-EDTA (GIBCO) supplemented with DNase I for 15 min at 37° C. Freshly sorted live single cells were counted and loaded onto the Chromium Controller (10× Genomics). Each sample was loaded into a separate channel on the Single Cell 3′ Chip targeting 3,000 cells. Libraries were prepared using the Chromium Single Cell 3′ Reagent kits v3 (10× Genomics) following the manufacturer's recommendations. The multiplexed libraries were loaded on a NovaSeq 6000 (Illumina) and paired-end reads were produced using a 200-cycle kit. Each sample was sequenced to an average depth of ~40,000 read pairs per cell.

Single-cell RNA-sequencing data bioinformatic analysis. The Cell Ranger pipeline was used to process the raw sequencing data, producing count matrices initially containing 3,509 cells in the Control group and 3,576 cells in the Doxo group. Package Seurat v4 was used in R to perform downstream analyses. Gene signature scores for the “Basal signature”, “Luminal signature”, “Myoepithelial signature” and “TNF signaling activity” were calculated using the AddModuleScore function in Seurat. The “TNF signaling activity” is composed of the TNF signaling genes in the HALLMARK_TNFA_SIGNALING_VIA_NFKB gene set. Single-cell level prediction of differentiation states in tumor organoid cells was performed in R using the CytoTRACE package publicly available. Single-sample enrichment of the “Normal Basal Signature,” “Normal Luminal Signature” and the dbEMT gene sets were analyzed using ssGSEA as implemented in the escape package. Signatures genes (top 200 DE genes for each lineage) for basal and luminal cells from normal mammary glands were obtained from publicly available RNA-seq data as described in the previous study.

Single-cell gene expression analysis. Single-cell gene-expression analysis of normal human breast was done using Fluidigm's M48 quantitative PCR (qPCR) DynamicArray microfluidic chips (Fluidigm) as previously described.

Deconvolution of bulk breast tumors and survival analysis. CIBERSORTx was used to deconvolve cell type abundances from microarray gene expression data of bulk breast tumors from the METABRIC dataset and Hatzis dataset. Default parameters as described in the “Tutorial” page were used to generate a signature matrix from the scRNA-seq dataset of breast cancers. Quantile normalization was run on microarray data, and bulk-mode batch correction (B-mode) was applied for cross-platform deconvolution. The R package survival (v3.1.12) was applied to determine the correlation between BCL11B⁺ epithelial tumor cells and relapse-free survival. The samples were stratified into two groups, “high” and “low,” based on if the abundance of the deconvolved BCL11B⁺ tumor population was more or less than the median. A Cox proportional hazard model was then employed to calculate the effect of the abundance of the deconvolved cell population on Relapse-free survival or Distant recurrence-free survival. Patients were censored at 60 months for the survival analysis.

Immunofluorescence. Normal mammary tissues or tumor samples were fixed in 4% formaldehyde solution and subjected to either paraffin or O.C.T. embedding (Tissue-Tek). Paraffin sections were de-paraffinized, dehydrated, and microwaved for 15 min at 95° C. in Sodium Citrate Buffer (10 mM Sodium Citrate, 0.05% Tween 20, pH 6.0) for antigen retrieval. Frozen sections were first hydrated with PBS for 10 min at room temperature and then microwaved for 15 min at 95° C. in Sodium Citrate Buffer for antigen retrieval. Tissue sections were blocked with staining buffer (PBS+5% BSA+0.1% Triton X-100) for 1 hour at room temperature and then incubated overnight at 4° C. with the primary antibodies diluted in the staining buffer. Samples were subsequently washed with PBS and incubated with conjugated secondary antibodies (Invitrogen) at 1:300 in staining buffer+DAPI (1 μg/ml) for 1 hour at room temperature. After PBS washing, tissue sections were mounted with Fluoromount® Aqueous Mounting Medium (Sigma). Images were acquired by a Carl Zeiss LSM 710 Meta confocal microscope or a Keyence fluorescence microscope. For whole-mount organoid staining, pre-chilled cell recovery solution (Corning) was added to the well for 20 min on ice to retrieve the organoids. The organoids were then transferred to a microcentrifuge tube and pelleted by quick spinning using a mini centrifuge. After being fixed in 4% formaldehyde solution for 20 min, the recovered organoids were permeabilized and blocked in organoid staining buffer (PBS+5% BSA+1% Triton X-100) for 1 hour at room temperature, followed by overnight incubation with primary antibodies diluted in the organoid staining buffer at 4° C. The organoids were then washed with PBS and incubated with conjugated secondary antibodies (Invitrogen) at 1:300 in organoid staining buffer+DAPI (1 μg/ml) for 1 hour at room temperature. After PBS washing, the organoids were mounted on a glass slide for immunofluorescence imaging.

Western blot. Cells were lysed in ice-cold RIPA buffer containing 1× Halt Protease and Phosphatase inhibitor cocktails (ThermoFisher Scientific) and boiled on a heat block at 100-degree for 15 min. Samples were loaded on a 4%-20% gradient SDS-PAGE gel (Bio-Rad) and run at 80v for 90 min and transferred onto PVDF membranes. Membranes were blocked with PBST+5% Non-fat dry milk for 1 hour at room temperature and then incubated with primary antibodies at 4° C. overnight. Membranes were subsequently washed by PBST and stained with HRP-conjugated secondary antibodies. Membranes were then washed and visualized using the SuperSignal™ West Dura Extended Duration Substrate (ThermoFisher Scientific).

Data and code availability. All data generated or analyzed during this study are included in the published article. The single-cell and bulk RNA-sequencing data generated in this study have been deposited in the Gene Expression Omnibus with the primary accession code GSE228442. Previously published scRNA-seq data that were reanalyzed here are available at GEO under accession codes GSE113196, GSE109774 and GSE176078. Previously published bulk RNA-seq and microarray expression data reanalyzed here are available at GEO under accession codes GSE87455 and GSE6883. All bioinformatics tools used in this study are publicly available.

Quantification and statistical analysis. Graphs are represented as bars showing standard error (SEM). P-values are calculated using paired/unpaired t-test and ANOVA test. Survival was measured using the Kaplan-Meier method. Each experiment has been replicated for at least 3 times. For animal studies, sample size was not predetermined to ensure adequate power to detect a pre-specified effect size, no animals were excluded from analyses, experiments were not randomized and investigators were not blinded to allocation during experiments.

### Example 2

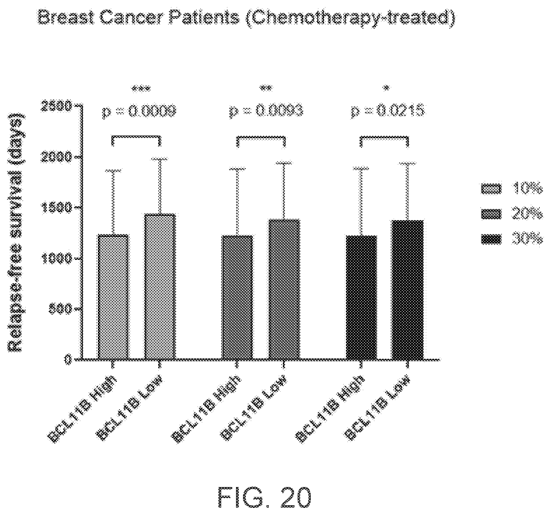

In normal breast epithelium, the percentage of BCL11B+ epithelial cells is approximately 3%, as determined by single-cell RNA-seq analysis. A subset of breast tumors demonstrate increased BCL11B expression, and analysis of tissue microarrays reveals that the proportion of BCL11B+ tumor cells (BCL11B+ tumor cells/all tumor cells) in these patients is generally higher than 10 percent. By setting different thresholds to separate between patients with high and low levels of BCL11B for survival analysis, it is apparent that individuals who have received chemotherapy and have over 10% or 20% of BCL11B tumor cells are significantly correlated with a poorer prognosis. Utilizing a threshold of 10% or 20% effectively separates the patients, as the differences in outcomes between the groups are most significant, see FIG. 20. Therefore, we conclude that patients with higher than 10-20% of BCL11B+ tumor cells are associated with a higher risk of resistance when receiving chemotherapy.

### REFERENCES

De Conti, G., Dias, M.H. & Bernards, R. Fighting Drug Resistance through the Targeting of Drug-Tolerant Persister Cells. Cancers 13 (2021).
Vasan, N., Baselga, J. & Hyman, D.M. A view on drug resistance in cancer. Nature 575, 299-309 (2019).
Hangauer, M. J. et al. Drug-tolerant persister cancer cells are vulnerable to GPX4 inhibition. Nature 551, 247-250 (2017).
Rehman, S. K. et al. Colorectal Cancer Cells Enter a Diapause-like DTP State to Survive Chemotherapy. Cell 184, 226-242 e221 (2021).
Echeverria, G. V. et al. Resistance to neoadjuvant chemotherapy in triple-negative breast cancer mediated by a reversible drug-tolerant state. Sci Transl Med 11 (2019).
Dhimolea, E. et al. An Embryonic Diapause-like Adaptation with Suppressed Myc Activity Enables Tumor Treatment Persistence. Cancer Cell 39, 240-256 e211 (2021).
Weber, W. A. Assessing tumor response to therapy. J Nucl Med 50 Suppl 1, 1S-10S (2009).
Liedtke, C. et al. Response to neoadjuvant therapy and long-term survival in patients with triple-negative breast cancer. J Clin Oncol 26, 1275-1281 (2008).
Prasetyanti, P.R. & Medema, J. P. Intra-tumor heterogeneity from a cancer stem cell perspective. Mol Cancer 16, 41 (2017).
Alvarez-Varela, A. et al. Mex3a marks drug-tolerant persister colorectal cancer cells that mediate relapse after chemotherapy. Nat Cancer (2022).
Ohta, Y. et al. Cell-matrix interface regulates dormancy in human colon cancer stem cells. Nature 608, 784-794 (2022).
Polyak, K. Heterogeneity in breast cancer. J Clin Invest 121, 3786-3788 (2011).
Perou, C. M. et al. Molecular portraits of human breast tumours. Nature 406, 747-752 (2000).

Sorlie, T. et al. Gene expression patterns of breast carcinomas distinguish tumor subclasses with clinical implications. Proc Natl Acad Sci USA 98, 10869-10874 (2001).
Turner, K. M., Yeo, S. K., Holm, T. M., Shaughnessy, E. & Guan, J. L. Heterogeneity within molecular subtypes of breast cancer. Am J Physiol Cell Physiol 321, C343-C354 (2021).
Fu, N. Y., Nolan, E., Lindeman, G. J. & Visvader, J. E. Stem Cells and the Differentiation Hierarchy in Mammary Gland Development. Physiol Rev 100, 489-523 (2020).
Van Keymeulen, A. et al. Distinct stem cells contribute to mammary gland development and maintenance. Nature 479, 189-193 (2011).
Rios, A. C., Fu, N. Y., Lindeman, G. J. & Visvader, J. E. In situ identification of bipotent stem cells in the mammary gland. Nature 506, 322-327 (2014).
Singh, A. & Settleman, J. E M T, cancer stem cells and drug resistance: an emerging axis of evil in the war on cancer. Oncogene 29, 4741-4751 (2010).
Voulgari, A. & Pintzas, A. Epithelial-mesenchymal transition in cancer metastasis: mechanisms, markers and strategies to overcome drug resistance in the clinic. Biochim Biophys Acta 1796, 75-90 (2009).
Bill, R. & Christofori, G. The relevance of EMT in breast cancer metastasis: Correlation or causality? FEBS Lett 589, 1577-1587 (2015).
Sikandar, S. S. et al. Role of epithelial to mesenchymal transition associated genes in mammary gland regeneration and breast tumorigenesis. Nat Commun 8, 1669 (2017).
Cai, S. et al. A Quiescent Bcl11b High Stem Cell Population Is Required for Maintenance of the Mammary Gland. Cell Stem Cell 20, 247-260 e245 (2017).
Curtis, C. et al. The genomic and transcriptomic architecture of 2,000 breast tumours reveals novel subgroups. Nature 486, 346-352 (2012).
Teschendorff, A.E., Miremadi, A., Pinder, S.E., Ellis, I.O. & Caldas, C. An immune response gene expression module identifies a good prognosis subtype in estrogen receptor negative breast cancer. Genome Biol 8, R157 (2007).
Wu, S. Z. et al. A single-cell and spatially resolved atlas of human breast cancers. Nat Genet 53, 1334-1347 (2021).
Hatzis, C. et al. A genomic predictor of response and survival following taxane-anthracycline chemotherapy for invasive breast cancer. JAMA 305, 1873-1881 (2011).
Swain, S. M., Shastry, M. & Hamilton, E. Targeting HER2-positive breast cancer: advances and future directions. Nat Rev Drug Discov 22, 101-126 (2023).
Lin, E. Y. et al. Progression to malignancy in the polyoma middle T oncoprotein mouse breast cancer model provides a reliable model for human diseases. Am J Pathol 163, 2113-2126 (2003).
Cheung, K. J., Gabrielson, E., Werb, Z. & Ewald, A. J. Collective invasion in breast cancer requires a conserved basal epithelial program. Cell 155, 1639-1651 (2013).
Cheung, K. J. et al. Polyclonal breast cancer metastases arise from collective dissemination of keratin 14-expressing tumor cell clusters. Proc Natl Acad Sci USA 113, E854-863 (2016).
Anampa, J., Makower, D. & Sparano, J. A. Progress in adjuvant chemotherapy for breast cancer: an overview. BMC Med 13, 195 (2015).
Sachs, N. et al. A Living Biobank of Breast Cancer Organoids Captures Disease Heterogeneity. Cell 172, 373-386 e310 (2018).
Cara, S. & Tannock, I. F. Retreatment of patients with the same chemotherapy: implications for clinical mechanisms of drug resistance. Ann Oncol 12, 23-27 (2001).
Januchowski, R., Wojtowicz, K. & Zabel, M. The role of aldehyde dehydrogenase (ALDH) in cancer drug resistance. Biomed Pharmacother 67, 669-680 (2013).
Luo, B., Yan, D., Yan, H. & Yuan, J. Cytochrome P450: Implications for human breast cancer. Oncol Lett 22, 548 (2021).
Hayes, J. D., Flanagan, J. U. & Jowsey, I. R. Glutathione transferases. Annu Rev Pharmacol Toxicol 45, 51-88 (2005).
Pal, B. et al. Single cell transcriptome atlas of mouse mammary epithelial cells across development. Breast Cancer Res 23, 69 (2021).
Giraddi, R. R. et al. Single-Cell Transcriptomes Distinguish Stem Cell State Changes and Lineage Specification Programs in Early Mammary Gland Development. Cell Rep 24, 1653-+ (2018).
Viswanathan, V. et al. Aldehyde dehydrogenase 3A1 deficiency leads to mitochondrial dysfunction and impacts salivary gland stem cell phenotype. PNAS Nexus 1, pgac056 (2022).
Gulati, G. S. et al. Single-cell transcriptional diversity is a hallmark of developmental potential. Science 367, 405-411 (2020).
Liu, R. et al. The prognostic role of a gene signature from tumorigenic breast-cancer cells. N Engl J Med 356, 217-226 (2007).
Abbas, T. & Dutta, A. p21 in cancer: intricate networks and multiple activities. Nat Rev Cancer 9, 400-414 (2009).
Mao, B., Zhang, Z. & Wang, G. BTG2: a rising star of tumor suppressors (review). Int J Oncol 46, 459-464 (2015).
Zhang, L., Yu, J., Park, B. H., Kinzler, K. W. & Vogelstein, B. Role of BAX in the apoptotic response to anticancer agents. Science 290, 989-992 (2000).
Ou, Y., Wang, S. J., Li, D., Chu, B. & Gu, W. Activation of SAT1 engages polyamine metabolism with p53-mediated ferroptotic responses. Proc Natl Acad Sci USA 113, E6806-E6812 (2016).
Tsai, Y. T. et al. ANGPTL4 Induces TMZ Resistance of Glioblastoma by Promoting Cancer Stemness Enrichment via the EGFR/AKT/4E-BP1 Cascade. Int J Mol Sci 20 (2019).
Khatib, A. et al. The glutathione peroxidase 8 (GPX8)/IL-6/STAT3 axis is essential in maintaining an aggressive breast cancer phenotype. Proc Natl Acad Sci USA 117, 21420-21431 (2020).
Jiang, J. et al. G Protein-Coupled Receptor GPR87 Promotes the Expansion of PDA Stem Cells through Activating JAK2/STAT3. Mol Ther Oncolytics 17, 384-393 (2020).
Misra, S. et al. Hyaluronan constitutively regulates activation of COX-2-mediated cell survival activity in intestinal epithelial and colon carcinoma cells. J Biol Chem 283, 14335-14344 (2008).
Long, S. et al. ECM1 regulates the resistance of colorectal cancer to 5-FU treatment by modulating apoptotic cell death and epithelial-mesenchymal transition induction. Front Pharmacol 13, 1005915 (2022).
Huang, S. L. & Chao, C. C. Silencing of Taxol-Sensitizer Genes in Cancer Cells: Lack of Sensitization Effects. Cancers (Basel) 7, 1052-1071 (2015).
Lanzi, C., Zaffaroni, N. & Cassinelli, G. Targeting Heparan Sulfate Proteoglycans and their Modifying Enzymes to Enhance Anticancer Chemotherapy Efficacy and Overcome Drug Resistance. Current Medicinal Chemistry 24, 2860-2886 (2017).
Dong, C., Li, P., Wu, Y., Guo, Z. & He, R. The 1q21.3 region driver gene EFNA3 promotes disease progression via inhibition of lung adenocarcinoma cell apoptosis. Transl Cancer Res 11, 1309-1320 (2022).
Sun, X. et al. HSPB1 as a novel regulator of ferroptotic cancer cell death. Oncogene 34, 5617-5625 (2015).
Ye, X. et al. Distinct EMT programs control normal mammary stem cells and tumour-initiating cells. Nature 525, 256-260 (2015).
Kröger, C. et al. Acquisition of a hybrid E/M state is essential for tumorigenicity of basal breast cancer cells (vol 116, pg 7353, 2019). Proceedings of the National Academy of Sciences of the United States of America 116, 11553-11554 (2019).
Varela, L. M. & Ip, M. M. Tumor necrosis factor-alpha: a multifunctional regulator of mammary gland development. Endocrinology 137, 4915-4924 (1996).
Wu, Y. & Zhou, B. P. TNF-alpha/NF-kappaB/Snail pathway in cancer cell migration and invasion. Br J Cancer 102, 639-644 (2010).
Askari, A. A. et al. Basal and inducible anti-inflammatory epoxygenase activity in endothelial cells. Biochem Biophys Res Commun 446, 633-637 (2014).
Endo, M., Masaki, T., Seike, M. & Yoshimatsu, H. TNF-alpha induces hepatic steatosis in mice by enhancing gene expression of sterol regulatory element binding protein-1c (SREBP-1c). Exp Biol Med (Maywood) 232, 614-621 (2007).
Zabala, M. et al. LEFTY1 Is a Dual-SMAD Inhibitor that Promotes Mammary Progenitor Growth and Tumorigenesis. Cell Stem Cell 27, 284-299 e288 (2020).
Tsherniak, A. et al. Defining a Cancer Dependency Map. Cell 170, 564-576 e516 (2017).
Yang, W. et al. Genomics of Drug Sensitivity in Cancer (GDSC): a resource for therapeutic biomarker discovery in cancer cells. Nucleic Acids Res 41, D955-961 (2013).
Kim, C. et al. Chemoresistance Evolution in Triple-Negative Breast Cancer Delineated by Single-Cell Sequencing. Cell 173, 879-893 e813 (2018).
Pastushenko, I. et al. Fat1 deletion promotes hybrid EMT state, tumour stemness and metastasis. Nature 589, 448-455 (2021).
Roberts, N. J., Zhou, S., Diaz, L. A., Jr. & Holdhoff, M. Systemic use of tumor necrosis factor alpha as an anticancer agent. Oncotarget 2, 739-751 (2011).
Kim, D., Paggi, J. M., Park, C., Bennett, C. & Salzberg, S. L. Graph-based genome alignment and genotyping with HISAT2 and HISAT-genotype. Nat Biotechnol 37, 907-915 (2019).
Anders, S., Pyl, P. T. & Huber, W. HTSeq—a Python framework to work with high-throughput sequencing data. Bioinformatics 31, 166-169 (2015).
Patro, R., Duggal, G., Love, M. I., Irizarry, R. A. & Kingsford, C. Salmon provides fast and bias-aware quantification of transcript expression. Nat Methods 14, 417-419 (2017).
Love, M. I., Huber, W. & Anders, S. Moderated estimation of fold change and dispersion for RNA-seq data with DESeq2. Genome Biol 15, 550 (2014).
Hao, Y. et al. Integrated analysis of multimodal single-cell data. Cell 184, 3573-3587 e3529 (2021).
Zhao, M., Kong, L., Liu, Y. & Qu, H. dbEMT: an epithelial-mesenchymal transition associated gene resource. Sci Rep 5, 11459 (2015).
Borcherding, N. et al. Mapping the immune environment in clear cell renal carcinoma by single-cell genomics. Commun Biol 4, 122 (2021).
Fu, N. Y. et al. EGF-mediated induction of Mcl-1 at the switch to lactation is essential for alveolar cell survival. Nature Cell Biology 17, 365-+ (2015).
Pal, B. et al. Construction of developmental lineage relationships in the mouse mammary gland by single-cell RNA profiling. Nat Commun 8, 1627 (2017).
Dalerba, P. et al. Single-cell dissection of transcriptional heterogeneity in human colon tumors. Nat Biotechnol 29, 1120-1127 (2011).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

What is claimed is:

1. A method for identifying an individual with breast cancer having increased risk of developing resistance to chemotherapy, the method comprising:
determining the prevalence of BCL11B expression in a sample population of breast cancer cells from the individual, where a prevalence of greater than about 10% BCL11B⁺ cells in the population identifies an individual with a higher risk of developing resistance when receiving chemotherapy.

2. The method of claim 1, further comprising treating an individual identified as having a higher risk of developing resistance when receiving chemotherapy, with a combination of an effective dose of a TNFα agonist, and a chemotherapeutic or immunotherapeutic agent.

3. The method of claim 2, wherein the sample population has a prevalence of greater than 15% BCL11B⁺ cells.

4. The method of claim 2, wherein the sample population has a prevalence of greater than 20% BCL11B⁺ cells.

5. The method of claim 2, wherein the TNFα agonist is recombinant human TNFα.

6. The method of claim 2, wherein the TNFα agonist is an antibody that activates one or both of TNFR1 and TNFR2.

7. The method of claim 2, wherein the chemotherapeutic agent is an effective dose of an anthracycline.

8. The method of claim 7, wherein the anthracycline is doxorubicin.

9. The method of claim 8, wherein doxorubicin is administered in combination with cyclophosphamide.

10. The method of claim 2, wherein the chemotherapeutic agent is an effective dose of a taxane.

11. The method of claim 1, wherein the sample is obtained prior to treatment of the cancer.

12. The method of claim 1, wherein the sample is obtained following treatment of the cancer.

13. The method of claim 1, wherein determining the prevalence of BCL11B expression in a population of breast cancer cells comprises:
obtaining a sample of cancer cells from the individual;
contacting the cancer cells with an affinity reagent specific for BCL11B; and
quantitating the number of cells positive for BCL11B expression.

14. The method of claim 13, wherein the affinity reagent is an antibody specific for BCL11B.

15. The method of claim 14, wherein quantitating is performed by one or more of flow cytometry, mass cytometry, confocal microscopy, and immunochemistry.

16. The method of claim 1, wherein determining the prevalence of BCL11B expression in a population of breast cancer cells comprises:
obtaining a sample of cancer cells from the individual;
quantitating by PCR the number of cells positive for BCL11B mRNA.

17. The method of claim 1, wherein determining the prevalence of BCL11B expression in a population of breast cancer cells comprises:
obtaining a sample of cancer cells from the individual;
quantitating by single cell RNA sequence analysis the number of cells positive for BCL11B mRNA.

18. A kit for use in the method of claim 1.
